(19) United States
(12) Patent Application Publication
(10) Pub. No.: US 2010/0016333 A1
(43) Pub. Date: Jan. 21, 2010

(54) Once-a-day (RNA-Polymerase Inhibiting or phenazine) - dihydropteroate synthase inhibiting - dihydrofolate reductase inhibiting antibiotic pharmaceutical product, formulation thereof, and use thereof in treating infection caused by Methicillin-Resistant Staphylococcus aureus

(76) Inventors: Henry H. Flanner, Montgomery Village, MD (US); Donald Treacy, Woodbine, MD (US); Sanna Tolle-Sander, North Potomac, MD (US); Beth A. Burnside, Bethesda, MD (US); Edward M. Rudnic, North Potomac, MD (US)

Correspondence Address: Raymond E. Stauffer, Esq.;c/o Carella, Byrne, Bain, Gilfillan, Cecchi,, Stewart & Olstein, 5 Becker Farm Road, Roseland, NJ, 07068, US

(21) Appl. No.: 11/890,747

(22) Filed: Aug. 7, 2007

Related U.S. Application Data
(60) Provisional application No. 60/836,026, filed on Aug. 7, 2006.
(60) Provisional application No. 60/836,313, filed on Aug. 8, 2006.

(51) Int. Cl. A61K 31/496; A61P 31/04

(52) U.S. Cl. 514/254.11

(57) ABSTRACT

Disclosed are once-a-day antibiotic products for treating Methicillin-Resistant Staphylococcus aureus, or “MRSA,” the products comprising: a combination of at least three different antibiotics, wherein one of the at least three different antibiotics is selected from the group consisting of RNA-Polymerase Inhibiting antibiotics or Phenazine antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (alternatively any or all of the aforementioned RNA-Polymerase Inhibiting antibiotics, Dihydropteroate Synthase Inhibiting antibiotics, and Dihydrofolate Reductase Inhibiting antibiotics may be in the form of analogues, derivatives, polymorphs, metabolites, pro-drugs, salts, and/or hydrates of any of the foregoing); optionally in further combination with a resistance inhibitor, preferably a LexA protease cleavage inhibitor.

This application claims the priority of U.S. Provisional Application Ser. No. 60/836,026, filed Aug. 7, 2006; and also claims the priority of U.S. Provisional Application Ser. No. 60/836,313, filed Aug. 8, 2006; the disclosures of each of which are hereby incorporated by reference in their entireties.

This invention relates to a once-a-day antibiotic product, and to the use and formulation thereof. More specifically this invention relates to a once-a-day antibiotic product comprising: a combination of at least three different antibiotics, wherein one of the at least three different antibiotics is selected from the group consisting of RNA-Polymerase Inhibiting antibiotics, one of the at least three different antibiotics is selected from the group consisting of Dihydropteroate Synthase Inhibiting antibiotics, and one of the at least three different antibiotics is selected from the group consisting of Dihydrofolate Reductase Inhibiting antibiotics (alternatively any or all of the aforementioned RNA-Polymerase Inhibiting antibiotics, Dihydropteroate Synthase Inhibiting antibiotics, and Dihydrofolate Reductase Inhibiting antibiotics may be in the form of analogues, derivatives, polymorphs, metabolites, pro-drugs, salts, and/or hydrates of any of the foregoing), and to the use and formulation of such an antibiotic product. More specifically still this invention relates to a once-a-day antibiotic product comprising the aforementioned combination of antibiotics (or analogues, etc.), to the formulation thereof, and to the use thereof in treating bacterial infection in a patient or subject. In several embodiments the invention is directed to improving upon the eradication of antibiotic-resistant bacterial pathogens and/or to reducing the emergence of any further resistant bacterial pathogens, while using the product to treat bacterial infection in a patient or subject (e.g., an infectious bacterial pathogen such as Methicillin-Resistant Staphylococcus aureus, or “MRSA”). In still other embodiments, the above-described invention is directed to a once-a-day antibiotic product comprising the aforementioned combination of antibiotics (or analogues, etc.), in further combination with a resistance inhibitor—preferably a LexA protease cleavage inhibitor; and to the similarly above-described use, and formulation of such an antibiotic product. In preferred embodiments, the above-described invention is administered orally. However, in other embodiments, the above-described invention may be delivered by a multitude of pharmaceutically acceptable routes that are known in the art and described hereinbelow.

As known in the art and as referred to herein the terms “once-a-day,” “one-a-day,” “once daily,” and “Q.D.” shall denote that the product of the hereinabove-described and hereinbelow-described invention is to be administered only once during any given twenty-four hour period, after which no further product or composition is administered during that same given twenty-four hour period. As referred to hereinabove and hereinbelow, either of the terms “patient” or “subject” shall each individually denote any host of a bacterial infection, or any organism suspected of hosting a bacterial infection, including without limitation humans and animals. As referred to hereinabove and hereinbelow, the terms “to treat,” “treating,” or “treatment” (of) such patient or subject shall mean that the hereinabove-described and/or hereinbelow-described products and/or processes are administered to and/or practiced upon the patient or subject, but shall neither necessarily imply nor foreclose actual treatment of such patient or subject by a physician, clinician, investigator, parent, custodian, or other caregiver; yet may include any act of prescribing or otherwise directing that any of the hereinabove-described and/or hereinbelow-described products and/or processes are administered to and/or practiced upon the patient or subject, by any such person. Similarly, “to treat,” “treating,” or “treatment” (of) such patient or subject may include any act whereby the hereinabove-described and/or hereinbelow-described products and/or processes are administered to and/or practiced upon the patient or subject by the patient or subject himself/herself, or by an inanimate device or similar means.

Staphylococcus aureus, sometimes referred to simply as “staph,” or “staph A,” is a common bacterium typically found on the skin and/or in the nasal passages of healthy people. While the presence of Staphylococcus aureus on the skin and/or in the nasal passages is usually harmless to a person at those sites, “staph” infections can occur as a result of breaks in the skin, such as through abrasions, lacerations, and wounds; or by way of surgical procedures or catheterizations. If staph gets into the body it can cause minor skin and soft tissue infections, such as boils or pimples; or it can cause more serious conditions, such as pneumonia, empyema, blood infections, bacteremia, sepsis, osteomyelitis, pyomytosis, necrotizing fascititis, purpura fulminans, infections of the bones and joints, urinary tract infections, toxic shock syndrome, and even death.

Methicillin-Resistant Staphylococcus aureus is a bacterial pathogen resistant to certain antibiotics, such as methicillin and other beta-lactams, including oxacillin, penicillin, nafcillin, amoxicillin, and the cephalosporins. (See Dellit et al., Interim Guidelines for Evaluation & Management of Community-Associated Methicillin-Resistant Staphylococcus Aureus Skin and Soft Tissue Infections In Outpatient Settings, Sep. 2, 2004; Infectious Diseases Society of Washington, pages 1-14). Another source explains that “[α]ll MRSA are characterized genotypically by the presence of mecA, which encodes for altered penicillin binding proteins (PBPs) (PBP2A) on their cell walls. The low affinity binding of PBP2A to antistaphylococcal penicillins results phenotypically in resistance to all β-lactam antibiotics.” (See Bradley S., Staphylococcus Aureus Pneumonia: Emergence of MRSA in the Community, Semin Respir Crit Care Med. 2005; 26(6):643-649).

In accordance with a first broad aspect of the invention, the RNA-Polymerase Inhibiting antibiotic, Dihydropteroate Synthase Inhibiting antibiotic, and Dihydrofolate Reductase Inhibiting antibiotic pharmaceutical product is administered once-a-day in treating a patient or subject infected with Methicillin-Resistant Staphylococcus aureus (MRSA), an infection most traditionally acquired nosocomially—in a hospital, clinic, dialysis center, or other healthcare-associated setting; or through contact with persons associated with such healthcare settings (e.g., through healthcare employed or healthcare exposed family members). Nosocomially acquired MRSA is more commonly referred to as healthcare-acquired (or healthcare-associated) MRSA, or HA-MRSA.

In divers embodiments of the invention the RNA-Polymerase Inhibiting antibiotic, Dihydropteroate Synthase Inhibiting antibiotic, and Dihydrofolate Reductase Inhibiting antibiotic pharmaceutical product is administered once-a-day in treating a patient or subject infected with healthcare-acquired Methicillin-Resistant Staphylococcus aureus, or HA-MRSA.

In other divers embodiments of the invention the RNA-Polymerase Inhibiting antibiotic, Dihydropteroate Synthase Inhibiting antibiotic, and Dihydrofolate Reductase Inhibiting antibiotic pharmaceutical product is administered once-a-day in treating a patient or subject infected with community-acquired Methicillin-Resistant Staphylococcus aureus (CA-MRSA). Without limiting the scope of these, or any other, embodiments of the herein-described invention, Applicants note that MRSA has been generally described as being community-acquired (or community-associated) if the MRSA-positive specimen is obtained outside hospital settings or within 2 days of hospital admission, and if it was from a person who had not been hospitalized within 2 years before the date of MRSA isolation. (See Salmenlinna et al., Community-Acquired Methicillin-Resistant Staphylococcus aureus, Finland, Emerging Infectious Diseases, 2002 June; 8(6):602-7). Still, another source notes that patients developing infections within 48 to 72 hours of hospital (healthcare) admission are presumed to have acquired the infection from the community. (See Bradley S., Staphylococcus Aureus Pneumonia: Emergence of MRSA in the Community, Semin Respir Crit. Care Med. 2005; 26(6):643-649).

These art-recognized distinctions notwithstanding, however, as reported cases of CA-MRSA become more widespread in the healthcare environment, definitions based on exposure risk and/or location of acquisition may prove to be of less epidemiological value. Under current modes of susceptibility testing, an isolate is likely to be CA-MRSA if it is resistant to all beta-lactam antibiotics and susceptible to most other antibiotic classes including the aminoglycosides, lincosamides, sulfonamides, fluoroquinolones, and macrolides. In addition to being similarly resistant to the beta-lactams, HA-MRSA is also known to carry resistance elements for several other antibiotic classes, including macrolides, aminoglycosides, fluoroquinolones, tetracyclines, and sulfonamides. While CA-MRSA is generally more susceptible to each of gentamicin, clindamycin, ciprofloxacin, and erythromycin than is HA-MRSA, it is expected that as CA-MRSA becomes more common it will similarly acquire resistance genes that will make its detection by routine antibiotic susceptibility testing more difficult. (See Bradley S., Staphylococcus Aureus Pneumonia: Emergence of MRSA in the Community, Semin Respir Crit. Care Med. 2005; 26(6):643-649). Additionally, “the prevalence of in vitro resistance to non-beta-lactam antimicrobial agents may be increasing among MRSA strains associated with community transmission.” (See Gorwitz et al.; Strategies for Clinical Management of MRSA in the Community: Summary of an Experts' Meeting Convened by the Centers for Disease Control and Prevention; Department of Health and Human Services; March 2006).

On the skin and soft tissues Methicillin-Resistant Staphylococcus aureus produces symptoms no different from any other type of Staphylococcus aureus bacteria: the skin will appear red and inflamed around wound sites. Symptoms in serious cases may include fever, lethargy, and headache. Intravenous drug users and persons with long-term illnesses or who are immuno-suppressed are at increased risk for infection by MRSA. Other non-healthcare related populations most at risk for infection by CA-MRSA include prisoners, athletes in contact sports, school children, daycare attendees, military personnel, Pacific Islanders, Native Americans, Alaskan Natives, male homosexuals, and persons infected with HIV.

In the last several years the incidence of community acquired-MRSA (CA-MRSA) has risen dramatically, and it is becoming increasingly more common world-wide for MRSA infection to occur in the community setting. Salmenlinna et al. report that from 1997 to 1999 21% of all MRSA-positive persons hospitalized in Finland had community-acquired MRSA. (See Community-Acquired Methicillin-Resistant Staphylococcus aureus, Finland; Infectious Diseases Vol. 8, No. 6, June 2002). In Los Angeles, Calif. one local Emergency Room has recently reported that 64% of skin and soft-tissue infections (“SSTI”) were caused by MRSA in 2003-2004, versus 29% in 2001-2002. (See Community Associated Methicillin-Resistant Staphylococcus Aureus: an Emerging Infectious Disease in Los Angeles County, The Public's Health Volume 5, Number 7, pgs. 4-5; County of Los Angeles, Dept. of Health Services July-August 2005). Though there is little in the way of nationwide U.S. statistics regarding the actual incidence of CA-MRSA pneumonia, the art anticipates that “more cases of staphylococcus pneumonia, and CA-MRSA in particular, will be seen in addition to those populations that were traditionally at risk for S. aureus respiratory tract infection.” (See Bradley S., Staphylococcus Aureus Pneumonia: Emergence of MRSA in the Community, Semin Respir Crit. Care Med. 2005; 26(6):643-649). As many strains contain cytotoxins that exacerbate the necrotizing (cell death) aspects of the infections, and hence the severity of the illnesses, and/or the rates of mortality of the illnesses, the development of effective, reliable antibiotic treatments for both CA-MRSA and HA-MRSA should be of paramount importance to their overall epidemiology.

In the treatment of bacterial infections, antibiotics, such as those commonly-used for treating MRSA, are generally dosed in formulations that require multiple administrations over the course of any given 24-hour period. As is known in the art, such dosing regimens may be twice-a-day (b.i.d.), whereby the formulation is administered every 12 hours; three times daily (t.i.d.), whereby the formulation is administered every 8 hours; four times daily (q.i.d.), whereby the formulation is administered every 6 hours; or such dosing regimens may even conceive of dosing the formulation in excess of four administrations per day. Repeated administrations of a drug throughout a 24-hour period may be disruptive to meals or sleep, thereby presenting a significant inconvenience for patients. In the treatment of elderly or incapacitated patients multiple administration regimens can result in poor compliance, and hence an ineffective treatment of the infection.

Much of the existing guidelines and art relating to antibiotic treatments for MRSA requires repeated administrations of antibiotics over the course of a 24-hour period (i.e., at least twice-a-day or more—b.i.d., t.i.d., or q.i.d dosing). Those of the existing guidelines and art relating to antibiotic treatments for MRSA that are touted for once daily administration are generally directed to the use of beta-lactams (i.e., cephalosporins, cephems, carbapenems, etc.,) an antibiotic class noted to have waning utility against MRSA; and/or are generally directed to injectable formulations.

U.S. Pat. Nos. 4,131,672 and 4,175,125 (Eli Lilly and Company) disclose halogenated phenylthioacetamido cephalosporanic acids and derivatives such as 7-[2′-(2″,5″-dichlorophenylthio)acetamido]cephalosporanic acid, as being effective antibiotics against Staphylococcus aureus cultures which show heterogeneous resistance to methicillin. Administration of this beta-lactam is preferably by intramuscular injection every four to six hours, or by oral tablet every four to six hours.

U.S. Pat. No. 5,334,590 (Merck & Co., Inc.) discloses the use of 2-phenyl-carbapenems against MRSA. The preferred method of administration of this beta-lactam is parenteral by i.v. infusion, i.v. bolus, or i.m. injection; 2, 3, or 4 times per day.

U.S. Pat. No. 5,789,584 (Microcide Pharmaceuticals, Inc.) discloses (7R)-7-(acylamino)-3-(substituted-pyridinyl)-3-cephem-4-carboxylic acids or their pharmacologically acceptable salts, as exhibiting antibiotic activity against MRSA. The preferred method of administration of the beta-lactam is intravenous or intraperitoneal, one to four times per day.

U.S. Pat. No. 6,265,394 (Bristol-Myers Squibb Company) discloses gram-positive cephem derivatives as being useful in the treatment of diseases caused by MRSA. The patent further discloses that in MRSA-infected mice, against a vancomycin control, the PD₅₀ (the dose of drug given which protects 50% of mice from mortality) may range from 0.8 to about 16.5 mg/kg when the beta-lactam is given by intramuscular injection twice within the post-infection 24-hour period.

U.S. Pat. No. 6,946,458 (University of South Florida) discloses N-thiolated beta-lactams, for use in the treatment or inhibition of MRSA, but the patent discloses nothing with regard to the daily frequency of dosing for treatment of this ever-more challenging to treat indication, with such beta-lactams.

The above referenced beta-lactam prior art to the contrary notwithstanding, however, the present and future utilities of beta-lactam oriented anti-MRSA regimens must be seriously questioned insofar as MRSA is generally recognized as being resistant to beta-lactams, including all penicillins and cephaolsporins—this, according to a recent multi-agency collaborative publication titled Interim Guidelines for Evaluation & Management of Community-Associated Methicillin-Resistant Staphylococcus Aureus Skin and Soft Tissue Infections in Outpatient Settings (Sep. 2, 2004, Infectious Diseases Society of Washington—Tacoma-Pierce County Health Department—Public Health Seattle & King County—Washington State Department of Health). Gorwitz et al. have also noted that HA-MRSA isolates “are usually resistant in vivo to multiple classes of antimicrobial agents,” and have further noted the resistance of CA-MRSA “to beta-lactams (the antimicrobial class that includes penicillins and cephalosporins) and macrolides/azalides (e.g., erythromycin, clarithromycin, azithromycin).” They additionally noted that “resistance to other classes of antimicrobial agents, such as fluoroquinolones and tetracyclines, occurs and may be increasing in prevalence.” (Gorwitz et al.; Strategies for Clinical Management of MRSA in the Community. Summary of an Experts'Meeting Convened by the Centers for Disease Control and Prevention; Department of Health and Human Services; March 2006). Accordingly, there is a need in the art for anti-MRSA formulations that are not reliant on beta-lactams, and that can provide the convenience of once-a-day dosing, preferably by way of oral administration.

The RNA-Polymerase Inhibiting antibiotics, Dihydropteroate Synthase Inhibiting antibiotics, and Dihydrofolate Reductase Inhibiting antibiotics are each individually known antibiotic classes, which are outside of the beta-lactam antibiotic class.

LexA is a protease found in bacteria, the cleavage of which protease is known in the art to promote mutations in response to DNA damage, leading to the development of the bacteria's resistance to antibiotics. Cirz et al. “have shown, in vivo, that preventing LexA cleavage renders bacteria unable to evolve resistance to either ciprofloxacin or rifampicin in a mouse thigh infection model.” (See Cirz et al.; Inhibition of Mutation and Combating the Evolution of Antibiotic Resistance; PLOS Biology June 2005/Vol. 3/Issue 6/e176, pages 1024-1033).

In accordance with an aspect of the invention there is provided a once-a-day, anti-MRSA pharmaceutical product providing a daily dosage of a combination of at least three different antibiotics, wherein one of the at least three different antibiotics is selected from the group consisting of RNA-Polymerase Inhibiting antibiotics, one of the at least three different antibiotics is selected from the group consisting of Dihydropteroate Synthase Inhibiting antibiotics, and one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing).

In general, in those embodiments containing only the three active ingredients selected from each of the RNA-Polymerase Inhibiting, Dihydropteroate Synthase Inhibiting, and Dihydrofolate Reductase Inhibiting antibiotic classes, each active ingredient accounts for about 5% to about 90% of the total compliment of active ingredient in the product. Preferably each active ingredient accounts for about 10% to about 85% of the total compliment of active ingredient in the product. More preferably each active ingredient accounts for about 15% to about 80% of the total compliment of active ingredient in the product.

In general, the individual antibiotics are present in the product in the following amounts: about 20 mg. to about 2000 mg. of an RNA-Polymerase Inhibiting antibiotic, about 100 mg. to about 3000 mg. of a Dihydropteroate Synthase Inhibiting antibiotic, and about 20 mg. to about 2000 mg. of a Dihydrofolate Reductase Inhibiting antibiotic.

In accordance with a further aspect of the invention there is provided a once-a-day anti-MRSA pharmaceutical product providing a daily dosage of a combination of at least three different antibiotics, wherein one of the at least three different antibiotics is selected from the group consisting of RNA-Polymerase Inhibiting antibiotics, one of the at least three different antibiotics is selected from the group consisting of Dihydropteroate Synthase Inhibiting antibiotics, and one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), in further combination with a daily dosage of a resistance inhibitor, preferably a LexA protease cleavage inhibitor.

In general, in those embodiments containing the three active ingredients selected from each of the RNA-Polymerase Inhibiting, Dihydropteroate Synthase Inhibiting, and Dihydrofolate Reductase Inhibiting antibiotic classes, and additionally containing a resistance inhibitor (such as a LexA protease cleavage inhibitor), each active ingredient accounts for about 2% to about 96% of the total compliment of active ingredient in the product. Preferably each active ingredient accounts for about 5% to about 90% of the total compliment of active ingredient in the product. More preferably each active ingredient accounts for about 10% to about 85% of the total compliment of active ingredient in the product.

In general, the combination of antibiotics and inhibitor are present in the product in the following amounts: about 20 mg. to about 2000 mg. of an RNA-Polymerase Inhibiting antibiotic, about 100 mg. to about 3000 mg. of a Dihydropteroate Synthase Inhibiting antibiotic, about 20 mg. to about 2000 mg. of a Dihydrofolate Reductase Inhibiting antibiotic, and about 5 mg. to about 5000 mg. of an inhibitor.

As non-limiting examples of the RNA-Polymerase Inhibiting antibiotics (analogues, derivatives, polymorphs, metabolites, pro-drugs, salts, and/or hydrates of any of the foregoing) that may be used in the invention there may be mentioned: rifamycin; rifampin; rifampicin; rifabutin; rifapentin; rifapentine; rifaximin; rifalazil; the ansamycin antibiotics (drug class); rifamycin SV; rifamycin B diethylamide; rifamycin W; rifamycin S; rifamycin P; rifamycin O; rifamycin R; rifamycin U; rifamycin Y; rifamycin 3-iminomethylenyl (—CH═N—) derivatives; rifamycin-imino-derivatives; rifamycin-C11-oxime derivatives; rifamycin-C11-oxime cyclo derivatives; spiro-rifamycin; C-25 carbamate rifamycin derivatives; rifamexil; rifamdin; rifamide; rifaprim; rifamet(h)oprim; kanglemycin A; protorifamycin; rifamycin verde; ansamycin LM427; rifamazine; streptolygidin; sorangicin A; MDL473; GE23077; other (bacterial) RNA-Polymerase inhibitors such as the CBR703 series (Artsimovitch I, et al., Science. 2003 Oct. 24; 302(5645):650-4); Microcin J25 (Mukhopadhyay et al. 2004 Mol Cell 14:739; Adelman et al. 2004 Mol Cell 14:753); and any antibiotic which may act synergistically with any, or each, of: a Dihydrofolate Reductase Inhibiting antibiotic (or analogue, derivative etc.) and/or a Dihydropteroate Synthase Inhibiting antibiotic (or analogue, derivative etc.).

In addition to the above-mentioned non-limiting examples, and consistent with the broadest reasonable interpretation of this specification (M.P.E.P. §2111), as used in the hereto-attached, or in any later-amended, claims the term RNA-Polymerase Inhibiting antibiotic is to be interpreted without regard to whether the manner by which any such antibiotic inhibits an RNA-Polymerase enzyme is allosteric or competitive.

Although one of ordinary skill in the art may or may not recognize saphenamycin as being an RNA-Polymerase Inhibiting antibiotic, in an alternative embodiment of the invention saphenamycin may be substituted for the RNA-Polymerase Inhibiting antibiotic of the instant invention, as such invention is hereinabove-described and hereinbelow-described. In an alternative embodiment of the invention a phenazine may be substituted for the RNA-Polymerase Inhibiting antibiotic of the instant invention, as such invention is hereinabove-described and hereinbelow-described. Saphenamycin is a phenazine and therefore, to the extent that saphneamycin is not an RNA-Polymerase Inhibiting antibiotic, saphenamycin is included in the alternative embodiment wherein a phenazine is used instead of an RNA-Polymerase Inhibiting antibiotic.

As non-limiting examples of the Dihydropteroate Synthase Inhibiting antibiotics (analogues, derivatives, polymorphs, metabolites, pro-drugs, salts, and/or hydrates of any of the foregoing) that may be used in the invention there may be mentioned: sulfamethoxazole; para-aminobenzenesulfonamide derivatives (aka sulfanilamide-analogues); sulfanilamide; sulfadiazine; sulfamethoxazole; sulfadoxine; sulfisoxazole; sulfisoxazole acetyl; sulfacetmide; sulfapyridine; sulfasalazine; mafenide; sulfadimethoxine; sulfaquinoxaline; silver sulfadiazine; mafenide acetate; sulfisoxazole diolamine; phtalylsulfacetamide; phtalylsulfathiazole; sulfaguanidine; sulfamazole; sulfamazone; sulfametopirazine; sulfametoxypiridazine; sulfametrol; succinylsulfathiazole; 2,4-Diaminoquinazolines; Pyridopyrimidines; 2,3-a-pyrimidines; 5-deaza-pteridine; Epiroprim; Brodimoprim; K-130; SRI 8858; the class of the sulfones (aka diaminophenylsulfones, such as dapsone and sulfoxone); and any antibiotic which may act synergistically with any, or each, of: a Dihydrofolate Reductase Inhibiting antibiotic (or analogue, derivative etc.), and/or a RNA-Polymerase Inhibiting antibiotic (or analogue, derivative etc.).

As non-limiting examples of the Dihydrofolate Reductase Inhibiting antibiotics (analogues, derivatives, polymorphs, metabolites, pro-drugs, salts, and/or hydrates of any of the foregoing) that may be used in the invention there may be mentioned: trimethoprim, pyrimethamine; iclaprim; ormetoprim; 2,4-diaminopyrimidine; sulfanilic acid; nitrosoisocytosines; monocyclic pteridines; proguanil; chloroguanide; cycloguanil; and any antibiotic which may act synergistically with any, or each, of: a Dihydropteroate Synthase Inhibiting antibiotic (or analogue, derivative etc.) and/or a RNA-Polymerase Inhibiting antibiotic (or analogue, derivative etc.).

As non-limiting examples of the resistance inhibitors that may be used in the invention there may be mentioned: any achaogen able to reduce the rate of induced mutagenesis, which may include nucleic acids, peptide nucleic acids, phages, phagemids, polypeptides, peptidomimetics, antibodies, small or large organic or inorganic molecules or any combination of the above; of natural or non-natural origin; able to bind to or interact with gene products that increase the rate of mutations in a cell or organism. Examples of such gene products include RecA, RecB, RecC, RecD, RecF, RecG, RecN, LexA, UmuC, UmuD, PolB, PolIV, Poly, PriA, RuvA, RuvB, RuvC, UmuC, UmuD, UvrA, UvrB, UvrD, or any homologs or analogs thereof. Examples of such polypeptides (and peptidomimetics thereof) include those comprising or consisting of dipeptide Ala-Ala, tripeptide Val-Ala-Ala, or SEQ ID NO: 1, 2, or 3. In some embodiments wherein the achaogen comprises an Ala-Gly bond, the bond may be modified so that it is not cleavable under normal physiological conditions. In some embodiments, the polypeptide or peptidomimetic is C-terminally modified, e.g., such that it is electrophilic. For further instruction on this aspect of the invention, the disclosures of U.S. Patent Application Publication 2006/0111302 A1 and PCT/US2004/039064 (WO 2005/056754 A2) are hereby incorporated by reference in their entireties.

Generally, the once-a-day product comprises: at least one component(s), wherein each component comprises a pharmaceutically acceptable carrier and at least one antibiotic(s); such that the product on whole, regardless of whether it contains one component or a plurality of components, further comprises: a combination of at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (alternatively any or all of the aforementioned RNA-Polymerase Inhibiting antibiotics, Dihydropteroate Synthase Inhibiting antibiotics, and Dihydrofolate Reductase Inhibiting antibiotics may be in the form of analogues, derivatives, polymorphs, metabolites, pro-drugs, salts, and/or hydrates of any of the foregoing); such that the product is administered to a patient once-a-day; and such that the product contains a therapeutically-effective, anti-MRSA daily dosage of an antibiotic combination comprising the aforementioned three different antibiotics, so selected from among the RNA-Polymerase Inhibiting, Dihydropteroate Synthase Inhibiting, and Dihydrofolate Reductase Inhibiting antibiotic classes. The at least one component(s) may be an immediate release component, a modified release component, or a combination of thereof. As used herein and as known in the art, immediate release components shall include any component from which the initiation of release, and/or the rate of release, of active ingredient is not substantially delayed, and/or slowed, and/or sustained, after administration of the product. As used herein and as known in the art, modified release components shall include any component which is not considered as hereinabove defined as an immediate release component. Non-limiting examples of such modified release components shall include: delayed release component(s), sustained (or extended) release component(s), and/or combinations of the foregoing. Accordingly, such sustained (or extended) release components may be formulated so that initiation of release of the individual antibiotic or combination of antibiotic(s) and/or inhibitor(s) therefrom is not substantially delayed after administration of the antibiotic product, or it may be formulated so that initiation of release of the individual antibiotic or combination of antibiotic(s) and/or inhibitor(s) therefrom is substantially delayed after administration of the antibiotic product. The formulating of the aforementioned components will be apparent to those of ordinary skill in the art in view of the disclosures herein, further guided by the disclosures of U.S. patent application Ser. Nos. 10/894,787; 10/894,786; 10/894,994; 10/917,059; 10/922,412; and 10/940,265; and by the disclosures of U.S. Pat. Nos. 6,544,555; 6,623,757; and 6,669,948; all of which are hereby incorporated by this reference in their entireties.

In some embodiments of the invention the product will contain a single component, wherein the single component comprises: either an immediate release component or a modified release component. In some embodiments of the invention the product will contain a plurality of components, wherein the plurality of components comprises: immediate release components; modified release components; or combinations of the foregoing. Regardless of whether the once-a-day product contains only a single component (modified in its release or not), or contains a plurality of components (modified in their release(s) or not); it is an aspect of every embodiment of the invention that the product as a whole contains a therapeutically-effective, anti-MRSA daily dosage of an antibiotic combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (alternatively any or all of the aforementioned RNA-Polymerase Inhibiting antibiotics, Dihydropteroate Synthase Inhibiting antibiotics, and Dihydrofolate Reductase Inhibiting antibiotics may be in the form of analogues, derivatives, polymorphs, metabolites, pro-drugs, salts, and/or hydrates of any of the foregoing). In some embodiments of the invention each of the at least three different antibiotics may be present in each component of the product. In other embodiments of the invention each of the at least three different antibiotics may be present in a separate component of the product either independent of, or to the exclusion of, the presence therein of any other, or all other, antibiotic(s). In yet other embodiments of the invention combinations of all, or less than all, of the at least three different antibiotics may be in all, or less than all, of the components of the product.

Consistent with the above-delimited component permutations, in several embodiments of the present invention there is provided a once-a-day, therapeutically-effective, anti-MRSA pharmaceutical product providing a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; wherein the product comprises a single component containing all three of the at least three different antibiotics so selected; wherein the single component is an immediate release component or a modified release component; and from which any of the initiation, rate, or duration, of the release of any of the at least three different antibiotics so selected may be the same or different as respectively compared to any of the initiation, rate, or duration, of the release of any other of the at least three different antibiotics so selected.

Similarly consistent with the above-delimited component permutations, in several embodiments of the present invention there is provided a once-a-day, therapeutically-effective, anti-MRSA pharmaceutical product providing a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; wherein the product comprises two components, one containing at least one of the at least three different antibiotics so selected and the other containing at least two of the at least three different antibiotics so selected; wherein the two components are each independently selected from the group consisting of immediate release components and modified release components; and from which any of the initiation, rate, or duration, of the release of any of the at least three different antibiotics so selected may be the same or different as respectively compared to any of the initiation, rate, or duration, of the release of any other of the at least three different antibiotics so selected, irrespective of either component. In several such embodiments both of the components are immediate release components. In several other such embodiments both of the components are delayed release components. In still several other such embodiments both of the components are sustained release components. In yet still several other such embodiments both of the components are a combination of any of immediate, delayed, and sustained release components.

Further consistent with the above-delimited component permutations, in several embodiments of the present invention there is provided a once-a-day, therapeutically-effective, anti-MRSA pharmaceutical product providing a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; wherein the product comprises three components each containing a different one of each of the at least three different antibiotics so selected; wherein the three components are each independently selected from the group consisting of immediate release components and modified release components; and from which any of the initiation, rate, or duration, of the release of any of the at least three different antibiotics so selected may be the same or different as respectively compared to any of the initiation, rate, or duration, of the release of any other of the at least three different antibiotics so selected, irrespective of any component. In several such embodiments all three components are immediate release components. In several other such embodiments all three components are delayed release components. In still several other such embodiments all three components are sustained release components. In yet still several other such embodiments all three components are a combination of any of immediate, delayed, and sustained release components.

In some embodiments of the present invention there is provided a once-a-day, therapeutically-effective, anti-MRSA pharmaceutical product providing a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; wherein the product comprises in any order at least three components: an immediate release component or a modified release component containing at least rifampin, an immediate release component or a modified release component containing at least sulfamethoxazole, and an immediate release component or a modified release component containing at least trimethoprim.

In some embodiments of the present invention there is provided a once-a-day, therapeutically-effective, anti-MRSA pharmaceutical product providing a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; wherein the product comprises an immediate release component that contains rifampin; and in either order, an immediate-release and/or subsequent modified-release component(s) containing sulfamethoxazole, and an immediate release and/or subsequent modified-release component(s) containing trimethoprim.

In another embodiment of the present invention there is provided a once-a-day, anti-MRSA pharmaceutical product, which is comprised of at least two, preferably at least three, components (at least one of which is a modified release component). Such components are formulated so that each of the components has a different release profile and so that the composition provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing).

In another embodiment of the present invention there is provided a once-a-day, anti-MRSA pharmaceutical product, which is comprised of at least two, preferably at least three, components (at least one of which is a modified release component). Such components are formulated so that each of the components has a different release profile and so that the composition provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), in further combination with a daily dosage of a resistance inhibitor, preferably a LexA protease cleavage inhibitor.

In another embodiment of the invention there is provided a once-a-day, anti-MRSA pharmaceutical product, wherein there are at least two, preferably at least three components (at least one of which is a modified release component), each of which has a different release profile, the release profile of each of the components being such that the components each start release of the individual antibiotic or combination of antibiotic(s) contained therein at different times after administration of the product, and the product provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing).

In another embodiment of the invention there is provided a once-a-day, anti-MRSA pharmaceutical product, wherein there are at least two, preferably at least three components (at least one of which is a modified release component), each of which has a different release profile, the release profile of each of the components being such that the components each start release of the individual antibiotic or combination of antibiotic(s) and/or inhibitor(s) contained therein at different times after administration of the product, and the product provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), in further combination with a daily dosage of a resistance inhibitor, preferably a LexA protease cleavage inhibitor.

Thus, in accordance with such embodiments of the present invention, there is provided a single or unitary antibiotic product that has contained therein at least two, preferably at least three components (at least one of which is a modified release component); each of which has a different release profile, whereby the individual antibiotic or combination of antibiotic(s) and/or inhibitor(s) contained in each of such components is released at different times, and the antibiotic product provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing); or provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), in further combination with a daily dosage of a resistance inhibitor, preferably a LexA protease cleavage inhibitor.

In accordance with another embodiment of the invention, the antibiotic product may be comprised of at least four different components, each of which has a different release profile, and the product provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing); or provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), in further combination with a daily dosage of a resistance inhibitor, preferably a LexA protease cleavage inhibitor.

In accordance with another embodiment of the invention, the antibiotic product may be comprised of at least five different components, each of which has a different release profile, and the product provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing); or provides a daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), in further combination with a daily dosage of a resistance inhibitor, preferably a LexA protease cleavage inhibitor.

In accordance with one embodiment of the invention, there are at least three components (at least one of which is a modified release component). One of the at least three components is an immediate release component whereby initiation of release of the individual antibiotic or combination of antibiotic(s) and/or inhibitor(s) therefrom is not substantially delayed after administration of the antibiotic product. The second and third of the at least three components are delayed release components (each of which may be a pH sensitive or a non-pH sensitive delayed component, depending on the type of antibiotic product), whereby the individual antibiotic or combination of antibiotic(s) and/or inhibitor(s) released therefrom is delayed until after initiation of release of the individual antibiotic or combination of antibiotic(s) and/or inhibitor(s) from the immediate release component.

In one embodiment, the second of the at least three components initiates release of the individual antibiotic or combination of antibiotic(s) and/or inhibitor(s) contained therein at least one hour after the first component, with the initiation of the release therefrom generally occurring no more than six hours after initiation of release of individual antibiotic or combination of antibiotic(s) and/or inhibitor(s) from the first component of the at least three components.

As hereinabove indicated, some embodiments of the antibiotic product may contain three, four, or more different components.

In a preferred embodiment, the antibiotic product is a once-a-day product, whereby after administration of the antibiotic product, no further composition is administered during the day; i.e., the preferred regimen is that the product is administered only once over a twenty-four hour period. Thus, in accordance with this preferred embodiment, there is a single administration of an antibiotic product with the antibiotic combination (and the optional inhibitor). The term single administration means that the total daily dosage of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), with or without the further combination of a daily dosage of a resistance inhibitor, preferably a LexA protease cleavage inhibitor, administered over a twenty-four hour period, is administered at the same time, which can be a single tablet or capsule or sachet or two or more thereof, provided that they are administered at essentially the same time.

In general, each of the components in the antibiotic product may have one or more individual antibiotics or a combination of antibiotic(s) and/or inhibitor(s), and each of the components may have the same individual antibiotic or the same combination of antibiotic(s) and/or inhibitor(s) or a different individual antibiotic or a different combination of antibiotic(s) and/or inhibitor(s).

In several embodiments of the present invention there is provided a once-a-day, therapeutically-effective, anti-MRSA pharmaceutical product providing a daily dosage of a combination of the antibiotics rifampin, sulfamethoxazole, and trimethoprim (or analogues, derivatives, polymorphs, metabolites, pro-drugs, salts, and/or hydrates of any of the foregoing) wherein the product comprises: an immediate release component containing rifampin in a range of about 150 mg. to about 600 mg.; an immediate release component containing a combination of sulfamethoxazole and trimethoprim (SMX-TMP), wherein the sulfamethoxazole is present in a range of about 200 mg. to about 800 mg. and the trimethoprim is present in a range of about 40 mg. to about 160 mg.; a first (in time to release) delayed release component containing a combination of sulfamethoxazole and trimethoprim (SMX-TMP), wherein the sulfamethoxazole is present in a range of about 100 mg. to about 400 mg. and the trimethoprim is present in a range of about 20 mg. to about 80 mg.; and a second (in time to release) delayed release component containing a combination of sulfamethoxazole and trimethoprim (SMX-TMP), wherein the sulfamethoxazole is present in a range of about 100 mg. to about 400 mg. and the trimethprim is present in a range of about 20 mg. to about 80 mg.

In several embodiments of the present invention there is provided a once-a-day, therapeutically-effective, anti-MRSA pharmaceutical product providing a daily dosage of a combination of the antibiotics rifampin, sulfamethoxazole, and trimethoprim (or analogues, derivatives, polymorphs, metabolites, pro-drugs, salts, and/or hydrates of any of the foregoing) wherein the product comprises: an immediate release component containing rifampin in a range of about 150 mg. to about 600 mg.; an immediate release component containing a combination of sulfamethoxazole and trimethoprim (SMX-TMP), wherein the sulfamethoxazole is present in a range of about 200 mg. to about 800 mg. and the trimethoprim is present in a range of about 40 mg. to about 160 mg.; a delayed release component containing a combination of sulfamethoxazole and trimethoprim (SMX-TMP), wherein the sulfamethoxazole is present in a range of about 50 mg. to about 200 mg. and the trimethoprim is present in a range of about 10 mg. to about 30 mg.; and a sustained release component containing a combination of sulfamethoxazole and trimethoprim (SMX-TMP), wherein the sulfamethoxazole is present in a range of about 150 mg. to about 600 mg. and the trimethoprim is present in a range of about 30 mg. to about 90 mg.

In several embodiments of the present invention there is provided a once-a-day, therapeutically-effective, anti-MRSA pharmaceutical product providing a daily dosage of a combination of the antibiotics rifampin, sulfamethoxazole, and trimethoprim (or analogues, derivatives, polymorphs, metabolites, pro-drugs, salts, and/or hydrates of any of the foregoing) wherein the product comprises: an immediate release component containing rifampin in a range of about 150 mg. to about 600 mg.; a first (in time to release) delayed release component containing a combination of sulfamethoxazole and trimethoprim (SMX-TMP), wherein the sulfamethoxazole is present in a range of about 130 mg. to about 540 mg. and the trimethoprim is present in a range of about 20 mg. to about 80 mg.; a second (in time to release) delayed release component containing a combination of sulfamethoxazole and trimethoprim (SMX-TMP), wherein the sulfamethoxazole is present in a range of about 130 mg. to about 540 mg. and the trimethoprim is present in a range of about 20 mg. to about 80 mg.; and a sustained release component containing a combination of sulfamethoxazole and trimethoprim (SMX-TMP), wherein the sulfamethoxazole is present in a range of about 130 mg. to about 540 mg. and the trimethoprim is present in a range of about 20 mg. to about 80 mg.

As long as the product as a whole contains a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics, it is within the scope of the invention that the product may be comprised of various types of individual components or combinations of various types of individual components. Non-limiting embodiment examples may include: a single immediate release component, a single modified release component, a plurality of immediate release components, a plurality of modified release components, and a plurality of components comprising combinations of immediate release components and modified release components; wherein any of the here-mentioned modified release components can each be any of the aforementioned delayed release component(s), sustained (or extended) release component(s), and/or combinations of the foregoing. In some embodiments the product comprises an immediate release component in combination with a delayed release component. In some embodiments the product comprises an immediate release component in combination with a sustained release component. In some embodiments the product comprises a plurality of immediate release components. In some embodiments the product comprises a plurality of delayed release components. In some embodiments the product comprises a plurality of sustained release components. In some embodiments the product comprises combinations of the foregoing.

It is to be understood that when it is disclosed herein that a component initiates release after another component, such terminology means that the component is designed and is intended to produce such later initiated release. It is known in the art, however, notwithstanding such design and intent, that some “leakage” of antibiotic may occur. Such “leakage” is not “release” as used herein.

The antibiotic product of the present invention, as hereinabove described, may be formulated for administration by a variety of routes of administration. For example, the antibiotic product may be formulated in a way that is suitable for topical administration; administration in the eye or the ear; rectal or vaginal administration; as a nasal preparation; by inhalation; as an injectable; or for oral administration. In a preferred embodiment, the antibiotic product is formulated in a manner such that it is suitable for oral administration.

For example, in formulating the antibiotic product for topical administration, such as by application to the skin, the components, each of which contains an individual antibiotic or combination of antibiotic(s) and/or inhibitor(s), may be formulated for topical administration by including such components in an oil-in-water emulsion, or a water-in-oil emulsion. In such a formulation, an immediate release component may be in the continuous phase, and a delayed release component may be in a discontinuous phase. The formulation may also be produced in a manner for delivery of three components as hereinabove described. For example, there may be provided an oil-in-water-in-oil emulsion, with oil being a continuous phase that contains the immediate release component, water dispersed in the oil containing a first delayed release component, and oil dispersed in the water containing a third delayed release component. The topical formulation may contain penetration enhancers. The topical formulation may contain long-lasting polymers that are Generally Recognized As Safe (GRAS) in the various pharmacopoeial compendia, such as those used in topically applied sun-screens. The release of the various active ingredients of the invention may also be initiated by means of a mechanical device.

It is also within the scope of the invention to provide an antibiotic product in the form of a patch, which includes antibiotic components having different release profiles, as hereinabove described.

In addition, the antibiotic product may be formulated for use in the eye or ear or nose, for example, as a liquid emulsion. For example, the component may be coated with a hydrophobic polymer whereby a component is in the oil phase of the emulsion, and a component may be coated with hydrophilic polymer, whereby a component is in the water phase of the emulsion.

Furthermore, the antibiotic product may be formulated for rectal or vaginal administration, as known in the art. This may take the form of a cream, an emulsion, a suppository, or other dissolvable component similar to those used for topical administration.

In an embodiment, the antibiotic product is formulated in a manner suitable for oral administration. Thus, for a non-limiting example, for oral administration, each of the components may be used as a pellet or a particle, with a pellet or particle then being formed into a unitary pharmaceutical composition, for example, in a capsule, or embedded in a tablet, or suspended in a liquid for oral administration. Similarly, for a further non-limiting example, for oral administration, each of the components may be used as pluralities of pellets or particles, with pluralities of pellets or particles then being formed into a unitary pharmaceutical composition, for example, in a capsule, or embedded in a tablet, or suspended in a liquid for oral administration.

Alternatively, in formulating an oral delivery system, each of the components of the composition may be formulated as a single layer or multilayer tablet or similar unit, and each tablet or similar unit may be administered once-a-day, as individual tablets or similar units provided that they are administered at essentially the same time; or each of the tablets or similar components may be put into a capsule to produce a unitary antibiotic composition to be administered once-a-day. Thus, as a non-limiting example, a three component antibiotic composition may include a first component in the form of a tablet that is an immediate release tablet, and may also include two or more additional tablets, each of which provides for a delayed release or a sustained release of any or all of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics, as hereinabove described.

The formulation of an antibiotic product including at least three components with different or similar release profiles for different routes of administration is deemed to be within the skill of the art from the teachings herein. As known in the art, with respect to delayed release, the time of release can be controlled by a variety of mechanisms such as pH, coating thickness, choice of polymer, and combinations of the foregoing.

In formulating an antibiotic product in accordance with one embodiment of the invention, an immediate release component of the composition generally provides from about 10% to about 100% of the total dosage of individual antibiotic or combination of antibiotic(s) and/or inhibitor(s) to be delivered by the composition, with such immediate release component generally providing at least 25% of the total dosage of individual antibiotic or combination of antibiotic(s) and/or inhibitor(s) to be delivered by the composition. In many cases, an immediate release component provides from about 20% to about 70% of the total dosage of individual antibiotic or combination of antibiotic(s) and/or inhibitor(s) to be delivered by the composition; however, in some cases it may be desirable to have an immediate release component provide for about 25% to about 50% of the total dosage of individual antibiotic or combination of antibiotic(s) and/or inhibitor(s) to be delivered by the composition.

The remaining components deliver the remainder of the individual antibiotic or combination of antibiotic(s) and/or inhibitor(s). If more than one component is used each of the components may provide about equal amounts of individual antibiotic or combination of antibiotic(s) and/or inhibitor(s); however, they may also be formulated so as to provide different amounts.

In accordance with the present invention, each of the components contains a single individual antibiotic or a combination of antibiotic(s) and/or inhibitor(s); however, each of the components may contain more than one individual antibiotic or combination of antibiotic(s) and or inhibitor(s).

In one embodiment, where the composition contains one immediate release component and two modified release components, the immediate release component provides from 15% to 75% (preferably 20% to 65%), by weight, of the total individual antibiotic or combination of antibiotic(s) and/or inhibitor(s); where there are three modified release components, the immediate release component provides from 15% to 75%, by weight, of the total individual antibiotic or combination of antibiotic(s) and/or inhibitor(s); and where there are four modified release components, the immediate release component provides from 15% to 75%, by weight, of the total individual antibiotic or combination of antibiotic(s) and/or inhibitor(s).

With respect to the modified release components, where there are two modified release components, the first modified release component (the one released earlier in time) provides from 15% to 85%, by weight, of the total individual antibiotic or combination of antibiotic(s) and/or inhibitor(s) provided by the two modified release components with the second modified release component providing the remainder of the individual antibiotic or combination of antibiotic(s) and/or inhibitor(s).

Where there are three modified release components, the earliest released component provides 10% to 70% by weight of the total individual antibiotic or combination of antibiotic(s) and/or inhibitor(s) provided by the three modified release components, the next in time modified release component provides from 10% to 70%, by weight, of the individual antibiotic or combination of antibiotic(s) and/or inhibitor(s) provided by the three modified release components and the last in time provides the remainder of the individual antibiotic or combination of antibiotic(s) and/or inhibitor(s) provided by the three modified release components.

When there are four modified release components, the earliest modified release component provides from 10% to 70%, by weight, the next in time modified release component provides from 10% to 70%, by weight, the next in time modified release component provides from 10% to 70%, by weight, and the last in time modified release component provides from 10% to 70%, by weight, in each case of the total individual antibiotic or combination of antibiotic(s) and/or inhibitor(s).

### The Immediate Release Component

The immediate release portion of this system can be a mixture of ingredients that breaks down quickly after administration to release the individual antibiotic or combination of antibiotic(s) and/or inhibitor(s). This can take the form of either a discrete pellet or granule that is mixed in with, or compressed with, the other components.

The materials to be added to the individual antibiotic or combination of antibiotic(s) and/or inhibitor(s) for the immediate release component can be, but are not limited to, microcrystalline cellulose, corn starch, pregelatinized starch, potato starch, rice starch, sodium carboxymethyl starch, hydroxypropylcellulose, hydroxypropylmethylcellulose, hydroxyethylcellulose, ethylcellulose, chitosan, hydroxychitosan, hydroxymethylated chitosan, cross-linked chitosan, cross-linked hydroxymethyl chitosan, maltodextrin, mannitol, sorbitol, dextrose, maltose, fructose, glucose, levulose, sucrose, lactose, dicalcium phosphate, polyvinylpyrrolidone (PVP), acrylic acid derivatives (Carbopol, Eudragit, etc.), polyethylene glycols (PEG200-20000).

It may be useful to have these materials present in the range of 1.0 to 60% (W/W).

In addition, it may be useful to have other ingredients in this system to aid in the dissolution of the drugs, or the breakdown of the components after ingestion or administration, or drug stability or dissolution. These ingredients can be surfactants, such as sodium lauryl sulfate, sodium monoglycerate, sorbitan monooleate, polyoxyethylene sorbitan monooleate, glyceryl monostearate, glyceryl monooleate, glyceryl monobutyrate, one of the non-ionic surfactants such as the Pluronic line of surfactants, Vitamin E-TPGS or any other material with surface active properties, or any combination of the above. Further additives may be from the group of stabilizers and antioxidants, e.g. ascorbic acid and vitamin E, BHT; buffers like citric acid or phosphate salts; and disintegrants like crosslinked polyvinyl pyrrolidone, crosslinked hydroxypropylcelluose, sodium starch glycolate, or sodium carboxymethylcellulose.

These materials may be present in the range of 0.05-15% (W/W).

### The Non-pH Sensitive Delayed Release Component

The components in this composition are the same as the immediate release unit, but with additional polymers integrated into the composition, or as coatings over the pellet or granule.

Several methods to affect a delayed release with non-pH dependent polymers are known to those skilled in the art. These include soluble or erodible barrier systems, enzymatically degraded barrier systems, rupturable coating systems, and plugged capsule systems among others. These systems have been thoroughly described in the literature (see “A Review of Pulsatile Drug Delivery” by Bussemer and Bodmeier in the Winter 2001 issue of American Pharmaceutical Review) and the formulations and methods (described therein) for their manufacture are hereby incorporated by reference.

Materials that can be used to obtain a delay in release suitable for this component of the invention can be, but are not limited to, polyethylene glycol (PEG) with molecular weight above 4,000 daltons (Carbowax), Polyethylene Oxides (Polyox), waxes such as white wax or bees wax, paraffin, acrylic acid derivatives (Eudragit), propylene glycol, and ethylcellulose.

Typically these materials can be present in the range of 0.5-40% (W/W) of this component.

### The pH Sensitive (Enteric) Release Component

The components in this composition are the same as the immediate release component, but with additional polymers integrated into the composition, or as coatings over the pellet or granule or tablet.

The kind of materials useful for this purpose can be, but are not limited to, cellulose acetate pthalate, Eudragit L, Eudragit S, Eudragit FS, and other pthalate salts of cellulose derivatives.

These materials can be present in concentrations from 3-30% (W/W).

### Sustained Release Component

The components in this composition are the same as the immediate release component, but with additional polymers integrated into the composition, or as coatings over the pellet or granule.

The kind of materials useful for this purpose can be, but are not limited to, ethylcellulose; hydroxypropylmethylcellulose; hydroxypropylcellulose; hydroxyethylcellulose; carboxymethylcellulose; methylcellulose; nitrocellulose; Eudragit R; Eudragit RS; and Eudragit R L; Carbopol; polyethyleneoxide or polyethylene glycols with molecular weights in excess of 8,000 daltons.

These materials can be present in concentrations from 4-20% (W/W).

When it is desired to delay initiation of release of the sustained release component, an appropriate coating may be used to delay initiation of the sustained release, such as a pH sensitive or a non-pH sensitive coating.

### The Non-pH Sensitive Coating for Sustained Release Component

Materials that can be used to obtain a delay in release suitable for this component of the invention can be, but are not limited to, polyethylene glycol (PEG) with molecular weight above 4,000 daltons (Carbowax), polyethyleneoxides (Polyox), waxes such as white wax or bees wax, paraffin, acrylic acid derivatives (Eudragit RS or RL), cellulose acetate, and ethylcellulose.

Typically these materials can be present in the range of 0.5-40% (W/W) of this component. Preferably the materials are present in an amount just enough to provide the desired in vivo lag time and Tₘₐₓ.

### The pH Sensitive Coating for Sustained Release Component

The kind of materials useful for this purpose can be, but are not limited to, cellulose acetate phthalate, Eudragit L, Eudragit S, Eudragit FS, and other pthalate salts of cellulose derivatives.

These materials can be present in concentrations from 4-20% (W/W) or more. Preferably the materials are present in an amount just enough to provide the desired in vivo lag time and Tₘₐₓ.

As hereinabove indicated, the units comprising the antibiotic product of the present invention can be in the form of discrete pellets or particles contained in a capsule, or particles embedded in a tablet or suspended in a liquid suspension. Furthermore, mintablets may be combined with or without pellets or powders into a capsule or larger tablet form; the tablet may be multilayer coated with coating layers containing active ingredients and/or functional excipients (e.g. pH-dependent or pH-independent polymers as listed herein). Further examples are listed in U.S. Provisional Patent Application Ser. No. 60/755,043; U.S. patent application Ser. No. 11/173,929; and U.S. patent application Ser. No. 11/021,309; the disclosures of each of which are hereby incorporated by reference in their entireties.

The antibiotic product of the present invention may be administered, for example, by any of the following routes of administration: sublingual, transmucosal, transdermal, parenteral, etc., and preferably is administered orally. The composition includes a therapeutically effective amount of a combination comprising at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics (or analogues, derivatives, polymorphs, metabolites, pro-drugs, salts, and/or hydrates of any of the foregoing), with or without the further combination of a therapeutically effective amount of a resistance inhibitor, preferably a LexA protease cleavage inhibitor, which amounts may vary in relation to each other, and in relation to the number of times that the composition is to be delivered in a day. In preferred embodiments the product is to be administered once-a-day. The composition is administered to a patient or subject in an amount effective for treating infection caused by MRSA.

The RNA-Polymerase Inhibiting antibiotic, Dihydropteroate Synthase Inhibiting antibiotic, and Dihydrofolate Reductase Inhibiting antibiotic (and optional inhibitor) product should be administered for a sufficient amount of time to treat the MRSA infection. In one embodiment the RNA-Polymerase Inhibiting antibiotic, Dihydropteroate Synthase Inhibiting antibiotic, and Dihydrofolate Reductase Inhibiting antibiotic (and optional inhibitor) product is administered for about 10 days, but may vary depending on severity of infection and clinical response. The product may benefit from being administered with or without food.

The invention will be further described with respect to the following examples; however, the scope of the invention is not limited thereby. All percentages in this specification, unless otherwise specified, are by weight.

### EXAMPLES

In general, the product may consist of any of the following immediate release (IR), delayed release (DR), and/or sustained release (SR) components. The manufacture of each component is described below. The components are combined into unit products known to those skilled in the art but not limited to capsules, tablets or sachets, in the appropriate ratio to achieve the proper dose to be administered; the dose may be divided into one or multiple unit products of the same or varying composition.

Unit products containing varying amounts of IR, DR, and/or SR components can be manufactured by techniques known to those skilled in the art, but are not limited to:
compression using a suitable tablet press, such as a rotary tablet press or bilayer/trilayer/multilayer tablet press, or
filling into a capsule with a suitable encapsulator, or
filling into a sachet with a suitable filler.

As used in the following examples the term RNAPi/RIFA shall indicate any RNA-Polymerase Inhibiting antibiotic, or any rifamycin antibiotic (or any derivatives, analogues, etc. of any RNA-Polymerase Inhibiting antibiotic or of any rifamycin antibiotic) or any compounds that may act by the same mechanism of action, including but not limited to the non-limiting examples of RNA-Polymerase Inhibiting antibiotics as are listed and/or mentioned hereinabove and/or hereinbelow.

As used in the following examples the term SMX/TMP shall indicate the fixed combination of a Dihydropteroate Synthase Inhibiting antibiotic with a Dihydrofolate Reductase Inhibiting antibiotic (or the combination of any Dihydropteroate Synthase Inhibiting antibiotic with any derivative, analogue, etc. of a Dihydrofolate Reductase Inhibiting antibiotic, or the combination of any Dihydrofolate Reductase Inhibiting antibiotic with any derivative, analogue, etc. of a Dihydropteroate Synthase Inhibiting antibiotic, or the combination of any derivative, analogue, etc. of any Dihydropteroate Synthase Inhibiting antibiotic with any derivative, analogue, etc. of a Dihydrofolate Reductase Inhibiting antibiotic, or the combination of any other compounds that may act by the same respective mechanisms of action, including but not limited to the non-limiting examples of Dihydropteroate Synthase Inhibiting antibiotics and Dihydrofolate Reductase Inhibiting antibiotics as are listed and/or mentioned hereinabove and/or hereinbelow. Preferably, this combination consists of sulfamethoxazole and trimethoprim in the ratio 5:1, but is not limited to this specific ratio or to these specific compounds.

### I. IR Components

Mix the ingredients in a suitable pharmaceutical mixer or granulator such as a planetary mixer, high-shear granulator, fluid bed granulator, or extruder, in the presence of water or other solvent, or in a dry blend, optionally followed by dry granulation techniques (slugging, roller compaction) or other methods known to those skilled in the art. If water or other solvent is used, dry the blend in a suitable pharmaceutical drier, such as a vacuum oven or forced-air oven. The product may be milled, sieved or further granulated, and/or compressed using a suitable tablet press, such as a rotary tablet press.

| | | |
|---|---|---|
| 1. | | |
| Rifapentin | 65% | (W/W) |
| Microcrystalline Cellulose (e.g. Avicel | 20% | |
| PH101) | | |
| Polyvinyl Pyrrolidone (K30) | 10% | |
| Croscarmellose sodium | 5% | |
| 2. | | |
| RNAPi/RIFA | 55% | (W/W) |
| Microcrystalline Cellulose (e.g. Avicel | 25% | |
| PH101) | | |
| Polyvinyl Pyrrolidone (K30) | 10% | |
| Croscarmellose sodium | 10% | |
| 3. | | |
| Rifampin | 65% | (W/W) |
| Microcrystalline Cellulose (e.g. Avicel | 20% | |
| PH101) | | |
| Hydroxypropyl Cellulose (e.g. Klucel LF) | 10% | |
| Croscarmellose sodium 5% | | |
| 4. | | |
| RNAPi/RIFA | 75% | (W/W) |
| Polyethylene glycol 4000 | 10% | |
| Polyethylene glycol 2000 | 10% | |
| Hydroxypropyl Cellulose (e.g. Klucel LF) | 5% | |
| Vitamin E TPGS | 1% | |
| 5. | | |
| RNAPi/RIFA | 75% | (W/W) |
| Polyethylene glycol 8000 | 20% | |
| Polyvinyl Pyrrolidone (K30) | 5% | |
| 6. | | |
| SMX/TMP | 65% | (W/W) |
| Microcrystalline Cellulose (e.g. Avicel | 20% | |
| PH101) | | |
| Polyvinyl Pyrrolidone (K30) | 10% | |
| Croscarmellose sodium | 4.5% | |
| SLS | 0.5% | |
| 7. | | |
| Sulfamethoxazole/Trimethoprim 5:1 | 55% | (W/W) |
| Microcrystalline Cellulose (e.g. Avicel | 25% | |
| PH101) | | |
| Polyvinyl Pyrrolidone (K30) | 10% | |
| Croscarmellose sodium | 9% | |
| Polyoxyl 35 Castor Oil | 1% | |
| 8. | | |
| SMX/TMP | 65% | (W/W) |
| Microcrystalline Cellulose (e.g. Avicel | 20% | |
| PH101) | | |
| Hydroxypropyl Cellulose (e.g. Klucel LF) | 10% | |
| Croscarmellose sodium | 4% | |
| 9. | | |
| SMX/TMP | 75% | (W/W) |
| Polyethylene glycol 4000 | 10% | |
| Polyethylene glycol 2000 | 10% | |
| Hydroxypropyl Cellulose (e.g. Klucel LF) | 5% | |
| 10. | | |
| SMX/TMP | 75% | (W/W) |
| Polyethylene glycol 8000 | 20% | |
| Polyvinyl Pyrrolidone (K30) | 5% | |
| 11. | | |
| Rifapentin | 85% | (W/W) |
| Microcrystalline Cellulose (e.g. Avicel | 7.5% | |
| PH101) | | |
| Polyvinyl Pyrrolidone (K30) | 4% | |
| Crosscarmellose Sodium | 2.5% | |
| Polyoxyl 35 Castor Oil | 1% | |
| 12. | | |
| Sulfamethoxazole:Trimethoprim 5:1 | 85% | (W/W) |
| Microcrystalline Cellulose (e.g. Avicel | 7.5% | |
| PH101) | | |
| Polyvinyl Pyrrolidone (K30) | 4% | |
| Crosscarmellose Sodium | 2.5% | |
| Polyoxyl 35 Castor Oil | 1% | |
| 13. | | |
| RNAPi/RIFA | 95% | (W/W) |
| Polyvinyl Pyrrolidone (K30) | 4% | |
| Polysorbate 80 | 1% | |
| 14. | | |
| SMX/TMP | 95% | (W/W) |
| Polyvinyl Pyrrolidone (K30) | 4% | |
| Polysorbate 80 | 1% | |
| 15. | | |
| RNAPi/RIFA | 40% | (W/W) |
| Microcrystalline Cellulose (e.g. Avicel | 23% | |
| PH101) | | |
| Lactose Monohydrate | 20% | |
| Polyvinyl Pyrrolidone (K30) | 10% | |
| Crosscarmellose Sodium | 5% | |
| Polyoxyl 35 Castor Oil | 2% | |
| 16. | | |
| SMX/TMP | 40% | (W/W) |
| Microcrystalline Cellulose (e.g. Avicel | 23% | |
| PH101) | | |
| Lactose Monohydrate | 20% | |
| Polyvinyl Pyrrolidone (K30) | 10% | |
| Crosscarmellose Sodium | 5% | |
| Polyoxyl 35 Castor Oil | 2% | |
| 17. | | |
| Rifapentin | 70% | (W/W) |
| Polyethylene Gycol 2000 | 10% | |
| Microcrystalline Cellulose | 10% | |
| Polyvinyl Pyrrolidone (K30) | 8% | |
| Vitamin E TPGS | 2% | |
| 18. | | |
| Sulfamethoxazole:Trimethoprim 5:1 | 70% | (W/W) |
| Polyethylene Gycol 2000 | 10% | |
| Microcrystalline Cellulose | 10% | |
| Polyvinyl Pyrrolidone (K30) | 8% | |
| Vitamin E TPGS | 2% | |
| 19. | | |
| Rifampin | 27.1% | (W/W) |
| Sulfamethoxazole | 36.2% | |
| Trimethoprim | 7.2% | |
| Microcrystalline Cellulose | 18.1% | |
| Crosspovidone | 5.2% | |
| Polyvinyl Pyrrolidone K30 | 5.2% | |
| Sodium Lauryl Sulfate | 1.0% | |
| Magnesium Stearate | 0.0% | |
| 20. | | |
| Rifampin | 21.3% | (W/W) |
| Sulfamethoxazole | 42.7% | |
| Trimethoprim | 10.7% | |
| Polyethylene Glycol 2000 | 14.2% | |
| Crosspovidone | 5.0% | |
| Polyvinyl Pyrrolidone K30 | 5.0% | |
| Sodium Lauryl Sulfate | 1.0% | |

### II. Delayed Release Components

Immediate release core pellets or tablets manufactured in accordance with section 1 are coated with pH-dependent or pH-independent polymers to generate the delayed release components of the formulation per compositions listed in examples 21-32. These are examples of modified release components.

The coatings are applied by film coating techniques commonly known to those skilled in the art. The delayed release component is created by layering of polymers onto an active core. In general, the steps involve first making a coating dispersion or solution in organic or aqueous solvent. Second, the coating is applied at the proper conditions to produce an acceptably uniform film. This is done in a suitable coating apparatus such as a pan coater or a fluid bed wurster column coater. Optionally the product may be further cured if necessary.

In addition to coating techniques, matrix type enteric components may be formulated with ingredients of examples 33 to 42 by mixing the ingredients in a suitable pharmaceutical mixer or granulator such as a planetary mixer, high-shear granulator, fluid bed granulator, or extruder, in the presence of water or other solvent, or in a hot melt or dry granulation process. If water or other solvent was used, dry the blend in a suitable pharmaceutical drier, such as a vacuum oven or forced-air oven. Allow the product to cool. The product produced may be milled, sieved or further granulated, and compressed using a suitable tablet press, such as a rotary tablet press, or filled into capsules using a suitable capsule filler such as an MG2 Futura, or filled into sachets with a suitable filler.

One skilled in the art will realize that the coating level, polymer type, matrix agent type and level can be adjusted to obtain the desired release characteristics.

| | | |
|---|---|---|
| 21. | | |
| Core from Examples 1-20 | 65% | (W/W) |
| Eudragit L Polymer | 25% | |
| Talc | 8% | |
| Triethyl Citrate | 2% | |
| 22. | | |
| Core from Examples 1-20 | 60% | (W/W) |
| Citric Acid | 10% | |
| Polyvinyl Pyrrolidone (K30) | 5% | |
| Eudragit RS Polymer | 20% | |
| Talc | 4% | |
| Triethyl Citrate | 1% | |
| 23. | | |
| Core from Examples 1-20 | 93% | (W/W) |
| Cellulose Acetate | 6.75% | |
| Polyethylene Glycol 400 | 0.25% | |
| 24. | | |
| Core from Examples 1-20 | 65% | (W/W) |
| Cellulose Acetate Phthalate | 30% | |
| Triethyl Citrate | 5% | |
| 25. | | |
| Core from Examples 1-20 | 75% | (W/W) |
| Cellulose Acetate Phthalate | 20% | |
| Triacetin | 5% | |
| 26. | | |
| Core from Examples 1-20 | 65% | (W/W) |
| Eudragit FS Polymer | 28% | |
| Talc | 5% | |
| Triethyl Citrate | 2% | |
| 27. | | |
| Core from Examples 1-20 | 65% | (W/W) |
| Eudragit S Polymer | 28% | |
| Talc | 5% | |
| Triethyl Citrate | 2% | |
| 28. | | |
| Core from Examples 1-20 | 80% | (W/W) |
| Eudragit L Polymer | 12.5% | |
| Talc | 6% | |
| Triethyl Citrate | 1.5% | |
| 29. | | |
| Core from Examples 1-20 | 75% | (W/W) |
| Eudragit L Polymer | 20% | |
| Talc | 3.5% | |
| Triethyl Citrate | 1.5% | |
| 30. | | |
| Core from Examples 1-20 | 60% | (W/W) |
| Eudragit L Polymer | 35% | |
| Talc | 4% | |
| Triethyl Citrate | 1% | |
| 31. | | |
| Core from Examples 1-20 | 65% | (W/W) |
| Cellulose Acetate Pthalate | 32.5% | |
| Triethyl Citrate | 2.5% | |
| 32. | | |
| Core from Examples 1-20 | 82% | (W/W) |
| Hydroxypropyl Methylcellulose Acetate | 11% | |
| Succinate (e.g. Aqoat HF) | | |
| Talc | 3% | |
| Triethyl Citrate | 3.5% | |
| Sodium Lauryl Sulphate | 0.5% | |
| 33. | | |
| RNAPi/RIFA | 75% | (W/W) |
| Microcrystalline Cellulose (e.g. Avicel | 5% | |
| PH101) | | |
| Hydroxypropyl methylcellulose | 20% | |
| phthalate | | |
| 34. | | |
| RNAPi/RIFA | 60% | (W/W) |
| Lactose Monohydrate | 10% | |
| Eudragit L Polymer | 30% | |
| 35. | | |
| RNAPi/RIFA | 70% | (W/W) |
| Polyethylene glycol 4000 | 10% | |
| Cellulose acetate phthalate | 20% | |
| 36. | | |
| RNAPi/RIFA | 60% | (W/W) |
| Polyethylene glycol 2000 | 10% | |
| Lactose Monohydrate | 20% | |
| Eudragit L Polymer | 10% | |
| 37. | | |
| RNAPi/RIFA | 70% | (W/W) |
| Microcrystalline Cellulose (e.g. Avicel | 20% | |
| PH101) | | |
| Cellulose acetate phthalate | 10% | |
| 38. | | |
| SMX/TMP | 75% | (W/W) |
| Microcrystalline Cellulose (e.g. Avicel | 5% | |
| PH101) | | |
| Hydroxypropyl methylcellulose | 20% | |
| phthalate | | |
| 39. | | |
| SMX/TMP | 60% | (W/W) |
| Lactose Monohydrate | 10% | |
| Eudragit L Polymer | 30% | |
| 40. | | |
| SMX/TMP | 70% | (W/W) |
| Polyethylene glycol 4000 | 10% | |
| Cellulose acetate phthalate | 20% | |
| 41. | | |
| SMX/TMP | 60% | (W/W) |
| Polyethylene glycol 2000 | 10% | |
| Lactose Monohydrate | 20% | |
| Eudragit L Polymer | 10% | |
| 42. | | |
| SMX/TMP | 70% | (W/W) |
| Microcrystalline Cellulose (e.g. Avicel | 20% | |
| PH101) | | |
| Cellulose acetate phthalate | 10% | |

### III. Sustained Release Component

Examples 43-49 utilize film coating techniques commonly known to those skilled in the art to create the sustained release component by layering of such sustained release polymers onto an active core such that the desired release rate is obtained. These are examples of modified release components. In general, the steps involve first making a coating dispersion or solution in organic or aqueous solvent. Second, the coating is applied at the proper conditions to produce an acceptably uniform film. This is done in a suitable coating apparatus such as a pan coater or a fluid bed wurster column coater. Optionally the product may be further cured if necessary. Curing studies are recommended with sustained release membranes.

To create a matrix type sustained release component, formulate the ingredients of example 50-67 by mixing the ingredients in a suitable pharmaceutical mixer or granulator such as a planetary mixer, high-shear granulator, fluid bed granulator, or extruder, in the presence of water or other solvent, or in a hot melt process. If water or other solvent was used, dry the blend in a suitable pharmaceutical drier, such as a vacuum oven or forced-air oven. Allow the product to cool.

The product produced may be milled, sieved or granulated, and compressed using a suitable tablet press, such as a rotary tablet press, or filled into capsules using a suitable capsule filler such as an MG2 Futura.

One skilled in the art will realize that any of the coating level, polymer type, matrix agent type and level can be adjusted to obtain the desired release rate.

| | | |
|---|---|---|
| 43. | | |
| Core from Examples 1-20 | 75% | (W/W) |
| Ethylcellulose (e.g. Ethocel Std 7FP) | 20% | |
| HPC | 5% | |
| 44. | | |
| Core from Examples 1-20 | 80% | (W/W) |
| Eudragit RS Polymer | 10% | |
| Eudragit RL Polymer | 5% | |
| Talc | 3% | |
| Triethyl Citrate | 2% | |
| 45. | | |
| Core from Examples 1-20 | 90% | (W/W) |
| Ethylcellulose (e.g. Ethocel Std 7FP) | 9% | |
| Triacetin | 1% | |
| 46. | | |
| Core from Examples 1-20 | 90% | (W/W) |
| Surelease | 10% | |
| 47. | | |
| Core from Examples 1-20 | 85% | (W/W) |
| Kollicoat SR | 10% | |
| TBC | 5 | |
| 48. | | |
| Core from Examples 1-20 | 80% | (W/W) |
| Polyethylene glycol 8000 | 5% | |
| Eudragit RS Polymer | 15% | |
| 49. | | |
| Core from Examples 1-20 | 93% | (W/W) |
| Cellulose Acetate | 6.75% | |
| PEG 400 | 0.25% | |
| 50. | | |
| RNAPi/RIFA | 75% | (W/W) |
| Hydroxyethylcellulose | 10% | |
| Polyethylene glycol 4000 | 10% | |
| Hydroxypropyl Cellulose (e.g. Klucel LF) | 5% | |
| 51. | | |
| SMX/TMP | 75% | (W/W) |
| Hydroxyethylcellulose | 10% | |
| Polyethylene glycol 4000 | 10% | |
| Hydroxypropyl Cellulose (e.g. Klucel LF) | 5% | |
| 52. | | |
| RNAPi/RIFA | 75% | (W/W) |
| Lactose Monohydrate | 10% | |
| Polyvinyl Pyrrolidone (K30) | 10% | |
| Polyethylene glycol 2000 | 5% | |
| 53. | | |
| SMX/TMP | 75% | (W/W) |
| Lactose Monohydrate | 10% | |
| Polyvinyl Pyrrolidone (K30) | 10% | |
| Polyethylene glycol 2000 | 5% | |
| 54. | | |
| RNAPi/RIFA | 75% | (W/W) |
| Polyethylene glycol 4000 | 10% | |
| Polyvinyl Pyrrolidone (K30) | 10% | |
| Hydroxypropyl Cellulose (e.g. Klucel LF) | 5% | |
| 55. | | |
| SMX/TMP | 75% | (W/W) |
| Polyethylene glycol 4000 | 10% | |
| Polyvinyl Pyrrolidone (K30) | 10% | |
| Hydroxypropyl Cellulose (e.g. Klucel LF) | 5% | |
| 56. | | |
| RNAPi/RIFA | 75% | (W/W) |
| Lactose Monohydrate | 15% | |
| Polyethylene glycol 4000 | 5% | |
| Polyvinyl Pyrrolidone (K30) | 5% | |
| 57. | | |
| Sulfamethoxazole:Trimethoprim 5:1 | 75% | (W/W) |
| Lactose Monohydrate | 15% | |
| Polyethylene glycol 4000 | 5% | |
| Polyvinyl Pyrrolidone (K30) | 5% | |
| 58. | | |
| RNAPi/RIFA | 75% | (W/W) |
| Polyethylene Oxide (e.g. Polyox WSR 301) | 20% | |
| Hydroxypropyl Cellulose (e.g. Klucel LF) | 5% | |
| 59. | | |
| SMX/TMP | 75% | (W/W) |
| Polyethylene Oxide (e.g. Polyox WSR 301) | 20% | |
| Hydroxypropyl Cellulose (e.g. Klucel HF) | 4.5% | |
| Polyoxyl 35 Castor Oil | 0.5% | |
| 60. | | |
| RNAPi/RIFA | 55% | (W/W) |
| Polyethylene Oxide (e.g. Polyox WSR 301) | 40% | |
| Hydroxypropyl Cellulose (e.g. Klucel HF) | 5% | |
| 61. | | |
| SMX/TMP | 55% | (W/W) |
| Polyethylene Oxide (e.g. Polyox WSR 301) | 39.5% | |
| Hydroxypropyl Cellulose (e.g. Klucel HF) | 5% | |
| Polyoxyl 35 Castor Oil | 0.5% | |
| 62. | | |
| RNAPi/RIFA | 75% | (W/W) |
| Lactose Monohydrate | 5% | |
| Polyvinyl Pyrrolidone (K30) | 10% | |
| Hydroxypropyl methylcellulose (K4M) | 10% | |
| 63. | | |
| SMX/TMP | 75% | (W/W) |
| Lactose Monohydrate | 5% | |
| Polyvinyl Pyrrolidone (K30) | 10% | |
| Hydroxypropyl methylcellulose (K4M) | 10% | |
| 64. | | |
| RNAPi/RIFA | 75% | (W/W) |
| Polyethylene glycol 4000 | 10% | |
| Polyvinyl Pyrrolidone (K30) | 10% | |
| Hydroxypropyl Cellulose (e.g. Klucel HF) | 5% | |
| 65. | | |
| SMX/TMP | 75% | (W/W) |
| Polyethylene glycol 4000 | 10% | |
| Polyvinyl Pyrrolidone (K30) | 10% | |
| Hydroxypropyl Cellulose (e.g. Klucel HF) | 5% | |
| 66. | | |
| Rifapentin | 75% | (W/W) |
| Hydroxypropyl cellulose (e.g. Klucel GF) | 15% | |
| Polyvinyl Pyrrolidone (K30) | 8% | |
| Vitamin E TPGS | 2% | |
| 67. | | |
| Sulfamethoxazole:Trimethoprim 5:1 | 75% | (W/W) |
| Hydroxypropyl cellulose (e.g. Klucel GF) | 15% | |
| Polyvinyl Pyrrolidone (K30) | 8% | |
| Vitamin E TPGS | 2% | |

### Specific Example Formulations

### Combination 1

Combination 1 consists of one bilayer tablet unit product containing IR and SR portions as listed in the following table. The dose is administered in multiple units of 1, 2, 3 or more tablets as directed by a physician to treat MRSA infections.

| | Total Unit Dose | | |
|---|---|---|---|
| Drug | (mg) | IR | SR |
| Rifampin | 300 | 300 | — |
| Sulfamethoxazole | 400 | | 400 |
| Trimethoprim | 80 | | 80 |

### Product Combination 1 Concept.

Rifampin micronized in IR granulation (per Example # 3)
Sulfamethoxaxole/Trimethoprim SR granulation (per Example # 55)
Blend each granulation with magnesium stearate and subsequently compress into bilayer tablets

### Composition Per Bilayer Tablet 1:

| | %/bilayer | |
|---|---|---|
| | tab | TOTAL |
| Granulation #57 | | 57.5% |
| Sulfamethoxazole | 36.0% | |
| Trimethoprim | 7.2% | |
| Lactose Monohydrate | 8.6% | |
| Polyethylene glycol 4000 | 2.9% | |
| Polyvinyl Pyrrolidone (K30) | 2.9% | |
| Blend with Mag Stearate | 0.5% | 0.5% |
| Granulation # 3 | | 41.5% |
| Rifampin | 27.0% | |
| Microcrystalline Cellulose | 8.3% | |
| (e.g. Avicel PH101) | | |
| Hydroxypropyl Cellulose | 4.1% | |
| (e.g. Klucel LF) | | |
| Croscarmellose sodium | 2.1% | |
| Blend with Mag Stearate | 0.4% | 0.4% |
| TOTAL per bilayer tablet | 100.00% | 100.00% |

### Combination 2

Combination 2 consists of one unit product tablet containing IR and DR portions as listed in the following table. The dose is administered in multiple units of 1, 2, 3 or more tablets as directed by a physician to treat MRSA infections.

| | Total Dose | | | |
|---|---|---|---|---|
| Drug | (mg) | IR | DR-1 | DR-2 |
| Rifapentin | 300 | 300 | — | — |
| Sulfamethoxazole | 400 | 200 | 100 | 100 |
| Trimethoprim | 80 | 40 | 20 | 20 |

### Product Combination 2 Concept.

Rifapentin micronized in IR granulation (per example # 1)
Sulfamethoxazole/Trimethoprim granulation with solubilizer (per example #7)
coat Sulfamethoxazole/Trimethoprim IR granules for DR (per example # 26 and 27)
Blend with magnesium stearate and compact granules into tablets

### Composition Per Tablet 2

| | %/combo | |
|---|---|---|
| | tablets | TOTAL |
| 1 (IR) | | 31.3% |
| Rifapentin | 20.3% | |
| Microcrystalline Cellulose (e.g. Avicel | 6.3% | |
| PH101) | | |
| Polyvinyl Pyrrolidone (K30) | 3.1% | |
| Croscarmellose sodium | 1.6% | |
| 7 (IR portion) | | 29.6% |
| SMX/TMP 5:1 | | |
| Sulfamethoxazole | 13.5% | |
| Trimethoprim | 2.7% | |
| Microcrystalline Cellulose (e.g. Avicel | 7.4% | |
| PH101) | | |
| Polyvinyl Pyrrolidone (K30) | 3.0% | |
| Croscarmellose sodium | 2.7% | |
| Polyoxyl 35 Castor Oil | 0.3% | |
| 28 (DR-1 portion) | | 18.5% |
| Core from Example 7 | | |
| Sulfamethoxazole | 6.8% | |
| Trimethoprim | 1.4% | |
| Microcrystalline Cellulose (e.g. Avicel | 3.7% | |
| PH101) | | |
| Polyvinyl Pyrrolidone (K30) | 1.5% | |
| Croscarmellose sodium | 1.3% | |
| Polyoxyl 35 Castor Oil | 0.1% | |
| Eudragit L Polymer | 2.3% | |
| Talc | 1.1% | |
| Triethyl Citrate | 0.3% | |
| 29 (DR-2 portion) | | 19.7% |
| Core from Example 7 | | |
| Sulfamethoxazole | 6.8% | |
| Trimethoprim | 1.4% | |
| Microcrystalline Cellulose (e.g. Avicel | 3.7% | |
| PH101) | | |
| Polyvinyl Pyrrolidone (K30) | 1.5% | |
| Croscarmellose sodium | 1.3% | |
| Polyoxyl Castor Oil | 0.1% | |
| Eudragit L Polymer | 3.9% | |
| Talc | 0.7% | |
| Triethyl Citrate | 0.3% | |
| Blend with Mag Stearate | 1.0% | 1.0% |
| TOTAL | 100.0% | 100.0% |

### Combination 3

Combination 3 consists of one unit product tablet containing IR and DR portions as listed in the following table. The dose is administered in multiple units of 1, 2, 3 or more tablets as directed by a physician to treat MRSA infections.

| | Total Dose | | | |
|---|---|---|---|---|
| Drug | (mg) | IR | DR-1 | DR-2 |
| Rifapentin | 300 | 300 | — | — |
| Sulfamethoxazole | 400 | 200 | 100 | 100 |
| Trimethoprim | 80 | 40 | 20 | 20 |

### Product Combination 3 Concept:

Rifapentin micronized in IR granulation (per example # 11)
Sulfamethoxazole/Trimethoprim granulation with solubilizer (per example #12)
coat Sulfamethoxazole/Trimethoprim IR granules for DR (per example # 22 and 23)
Blend with magnesium stearate and Crosscarmellose Sodium and compact granules into tablets

### Composition Per Tablet 3

| | %/combo | |
|---|---|---|
| | tablet | TOTAL |
| Example 11(IR) | | 32.8% |
| Rifapentin | 27.9% | |
| Microcrystalline Cellulose (e.g. Avicel | 2.5% | |
| PH101) | | |
| Polyvinyl Pyrrolidone (K30) | 1.6% | |
| Croscarmellose sodium | 0.8% | |
| Example 12(IR portion) | | 26.3% |
| SMX/TMP 5:1 | | |
| Sulfamethoxazole | 18.6% | |
| Trimethoprim | 3.7% | |
| Microcrystalline Cellulose (e.g. Avicel | 2.0% | |
| PH101) | | |
| Polyvinyl Pyrrolidone (K30) | 1.1% | |
| Croscarmellose sodium | 0.7% | |
| Polyoxyl Castor Oil | 0.3% | |
| 28(DR-1 portion) | | 18.2% |
| Core from Example 7 80% (W/W) | | |
| Sulfamethoxazole | 9.3% | |
| Trimethoprim | 1.9% | |
| Microcrystalline Cellulose (e.g. Avicel | 1.0% | |
| PH101) | | |
| Polyvinyl Pyrrolidone (K30) | 0.5% | |
| Croscarmellose sodium | 0.3% | |
| Polyoxyl Castor Oil | 0.1% | |
| Eudragit L Polymer | 3.2% | |
| Talc | 1.5% | |
| Triethyl Citrate | 0.4% | |
| 29 (DR-2 portion) | | 19.9% |
| Core from Example 7 | | |
| Sulfamethoxazole | 9.3% | |
| Trimethoprim | 1.9% | |
| Microcrystalline Cellulose (e.g. Avicel | 1.0% | |
| PH101) | | |
| Polyvinyl Pyrrolidone (K30) | 0.5% | |
| Croscarmellose sodium | 0.3% | |
| Polyoxyl Castor Oil | 0.1% | |
| Eudragit L Polymer | 5.4% | |
| Talc | 0.9% | |
| Triethyl Citrate | 0.4% | |
| Blend all 4 granular portions with | | 2.7% |
| Crosscarmellose Sodium | 1.7% | |
| Mag Stearate | 1.0% | |
| TOTAL | 100.00% | 100.00% |

### Combination 4

Combination 4 consists of one unit product capsule containing IR and DR portions as listed in the following table. The dose is administered in multiple units of 1, 2, 3 or more capsules as directed by a physician to treat MRSA infections.

| | Total Dose | | |
|---|---|---|---|
| Drug | (mg) | IR | DR-1 |
| Rifampin | 250 | 250 | — |
| Sulfamethoxazole | 400 | 300 | 100 |
| Trimethoprim | 80 | 60 | 20 |

### Product Combination 4 Concept.

1 dose=2 capsules of equal composition: 1 DR minitab and IR granulation(s)
Rifampin micronized in IR granulation (per example# 11)
Sulfamethoxazole/Trimethoprim granulation with solubilizer (per example #12)
Compress Sulfamethoxazole/Trimethoprim IR minitabs with Mag Stearate
coat Sulfamethoxazole/Trimethoprim IR minitabs for DR
Blend IR granulations, fill coated minitab and granulation into capsule

| | %/combo | |
|---|---|---|
| | capsule | Total |
| Example 11 (IR) | | 32.8% |
| Rifapentin | 27.9% | |
| Microcrystalline Cellulose (e.g. | 2.5% | |
| Avicel PH101) | | |
| Polyvinyl Pyrrolidone (K30) | 1.6% | |
| Croscarmellose sodium | 0.8% | |
| Example12 (IR portion) | | 47.2% |
| SMX/TMP 5:1 | | |
| Sulfamethoxazole | 33.4% | |
| Trimethoprim | 6.7% | |
| Microcrystalline Cellulose (e.g. | 3.5% | |
| Avicel PH101) | | |
| Polyvinyl Pyrrolidone (K30) | 1.9% | |
| Croscarmellose sodium | 1.2% | |
| Polyoxyl Castor Oil | 0.5% | |
| (DR-1 portion) | | 20.1% |
| Sulfamethoxazole | 11.1% | |
| Trimethoprim | 2.2% | |
| Microcrystalline Cellulose (e.g. | 1.2% | |
| Avicel PH101) | | |
| Polyvinyl Pyrrolidone (K30) | 0.6% | |
| Croscarmellose sodium | 0.4% | |
| Polyoxyl Castor Oil | 0.2% | |
| Blend granulation with | | |
| Crosscarmellose Sodium | 0.6% | |
| Mag Stearate | 0.2% | |
| compress and coat with 7% wt gain on tab | | |
| cores | | |
| Eudragit L Polymer | 2.7% | |
| Talc | 0.6% | |
| Triethyl Citrate | 0.3% | |
| TOTAL | 100.00% | 100.00% |

### Combination 5

Combination 5 consists of one unit product tablet containing IR and SR portions as listed in the following table. The dose is administered in multiple units of 1, 2, 3 or more tablets as directed by a physician to treat MRSA infections.

| | Total Dose | | |
|---|---|---|---|
| Drug | (mg) | IR | SR |
| Rifapentin | 300 | 300 | — |
| Sulfamethoxazole | 400 | 200 | 200 |
| Trimethoprim | 80 | 40 | 40 |

### Product Combination 5 Concept.

Rifapentin in IR granulation (per example# 11)
Sulfamethoxazole/Trimethoprim IR granulation with solubilizer (per example # 12)
Sulfamethoxazole/Trimethoprim SR granulation with solubilizer (per example #57), blend with Magnesium Stearate
Blend IR granulations with Crosscarmellose Sodium and Magnesium Stearate
Compress into bilayer tablets

### Composition Tablet #5:

| | %/combo | |
|---|---|---|
| | tab | Total |
| Example 11(IR) | | 32.7% |
| Rifapentin | 27.8% | |
| Microcrystalline cellulose | 2.5% | |
| Povidone | 1.6% | |
| Croscarmellose sodium | 0.8% | |
| Example 12(IR portion) | | 28.1% |
| SMX/TMP 5:1 | | |
| Sulfamethoxazole | 18.5% | |
| Trimethoprim | 3.7% | |
| Microcrystalline cellulose | 2.9% | |
| Povidone | 1.6% | |
| Croscarmellose sodium | 1.0% | |
| Polyoxyl Castor Oil | 0.4% | |
| | 0.0% | |
| Blend granulation with | | 6.6% |
| Crosscarmellose Sodium | 1.5% | |
| Mag Stearate | 5.1% | |
| Example 59/SR | | 29.6% |
| SMX/TMP | | |
| Sulfamethoxazole | 18.5% | |
| Trimethoprim | 3.7% | |
| Polyethyleneoxide | 5.9% | |
| Hydroxypropylcellulose | 1.3% | |
| Polyoxyl 35 Castor Oil | 0.1% | |
| Blend granulation with | | 3.0% |
| Mag Stearate | 3.0% | |
| | 100.0% | 100.0% |

### Combination 6

Combination 6 consists of one unit product tablet containing IR portions as listed in the following table. The dose is administered in multiple units of 1, 2, 3 or more tablets as directed by a physician to treat MRSA infections.

| | Total IR Dose |
|---|---|
| Drug | (mg) |
| Rifapentin | 300 |
| Sulfamethoxazole | 400 |
| Trimethoprim | 80 |

### Product Combination 6 Concept:

Rifapentin in IR granulation (per example# 17)
Sulfamethoxazole/Trimethoprim IR granulation with solubilizer (per example #18)
Blend IR granulations with Crosscarmellose Sodium and Magnesium Stearate
Compress into tablets

| | %/combo | |
|---|---|---|
| | tab | Total |
| Example 17(IR) | | 36.1% |
| Rifapentin | 25.3% | |
| Polyethylene Glycol 2000 | 3.6% | |
| Microcrystalline cellulose | 3.6% | |
| Polyvinyl Pyrrolidone K30 | 2.9% | |
| Vitamin E TPGS | 0.7% | |
| Example 18(IR portion) | | 57.8% |
| SMX/TMP 5:1 | | |
| Sulfamethoxazole | 33.7% | |
| Trimethoprim | 6.7% | |
| Polyethylene Glycol 2000 | 5.8% | |
| Microcrystalline cellulose | 5.8% | |
| Polyvinyl Pyrrolidone K30 | 4.6% | |
| Vitamin E TPGS | 1.2% | |
| | 0.0% | |
| Blend granulation with | | 6.1% |
| Crosscarmellose Sodium | 5.1% | |
| Mag Stearate | 1.0% | |
| | 100.0% | 100.0% |

### Combination 7

Combination 7 consists of one unit product tablet containing SR portions as listed in the following table. The dose is administered in multiple units of 1, 2, 3 or more tablets as directed by a physician to treat MRSA infections.

| | Total SR Dose |
|---|---|
| Drug | (mg) |
| Rifapentin | 300 |
| Sulfamethoxazole | 400 |
| Trimethoprim | 80 |

### Product Combination 7 Concept:

Rifapentin in SR granulation (per example# 66)
Sulfamethoxazole/Trimethoprim SR granulation with solubilizer (per example #67)
Blend SR granulations with Magnesium Stearate
Compress into tablets

| | %/combo | |
|---|---|---|
| | tab | Total |
| Example 66(SR) | | 38.0% |
| Rifapentin | 28.5% | |
| Hydroxyproppyl Cellulose (Klucel | 5.7% | |
| GF) | | |
| Polyvinyl Pyrrolidone K30 | 3.0% | |
| Vitamin E TPGS | 0.8% | |
| Example 67(SR portion) | | 60.8% |
| SMX/TMP 5:1 75% (W/W) | | |
| Sulfamethoxazole | 38.0% | |
| Trimethoprim | 7.6% | |
| Hydroxyproppyl Cellulose (Klucel | 9.1% | |
| GF) | | |
| Polyvinyl Pyrrolidone K30 | 4.9% | |
| Vitamin E TPGS | 1.2% | |
| | 0.0% | |
| Blend granulations with | | 1.1% |
| Mag Stearate | 1.1% | |
| | 100.0% | 100.0% |

### Combination 8

Combination 8 consists of one unit product tablet containing IR portions as listed in the following table. The dose is administered in multiple units of 1, 2, 3 or more tablets as directed by a physician to treat MRSA infections.

| | Total IR Dose |
|---|---|
| Drug | (mg) |
| Rifampin | 300 |
| Sulfamethoxazole | 600 |
| Trimethoprim | 150 |

### Product Combination 8 Concept:

Blend all dry ingredients; add Magnesium Stearate and blend
Compress into tablets

| | % |
|---|---|
| Rifampin | 26.9 |
| Sulfamethoxazole | 35.8 |
| Trimethoprim | 7.2 |
| Microcrystalline | 17.9 |
| Cellulose | |
| Crosspovidone | 5.1 |
| Polyvinyl Pyrrolidone | 5.1 |
| K30 | |
| Sodium Lauryl Sulfate | 1.0 |
| Magnesium Stearate | 1.0 |
| | 100.0 |

### Combination 9

Combination 9 consists of one unit product tablet containing IR portions as listed in the following table. The dose is administered in multiple units of 1, 2, 3 or more tablets as directed by a physician to treat MRSA infections.

| | Total IR Dose |
|---|---|
| Drug | (mg) |
| Rifampin | 300 |
| Sulfamethoxazole | 600 |
| Trimethoprim | 150 |

| | % |
|---|---|
| Rifampin | 21.1 |
| Sulfamethoxazole | 42.3 |
| Trimethoprim | 10.6 |
| Polyethylene Glycol | 14.1 |
| 2000 | |
| Crosspovidone | 5.0 |
| Polyvinyl Pyrrolidone | 5.0 |
| K30 | |
| Sodium Lauryl Sulfate | 1.0 |
| Magnesium Stearate | 1.0 |
| | 100.0 |

### Further Examples

### Example Compositions

### See Table Below

| | Active | Ex 1 | Ex 2 | Ex 3 | Ex 4 | Ex 5 | Ex 6 | Ex 7 | Ex 8 | Ex 9 |
|---|---|---|---|---|---|---|---|---|---|---|
| Pulse | Ingredient | (mg) | (mg) | (mg) | (mg) | (mg) | (mg) | (mg) | (mg) | (mg) |
| IR | RNAPi/RIFA | 150 | 300 | 600 | 150 | 600 | 150 | 600 | 150 | 600 |
| | analogue | | | | | | | | | |
| IR | SMX | 200 | 400 | 800 | 200 | 800 | 200 | 800 | 0 | 0 |
| | TMP | 40 | 80 | 120 | 40 | 120 | 40 | 120 | 0 | 0 |
| DR-1 | SMX | 100 | 200 | 400 | 50 | 200 | 0 | 0 | 133.3 | 533.3 |
| | TMP | 20 | 40 | 60 | 10 | 30 | 0 | 0 | 26.6 | 80 |
| DR-2 | SMX | 100 | 200 | 400 | 0 | 0 | 50 | 200 | 133.3 | 533.3 |
| | TMP | 20 | 40 | 60 | 0 | 0 | 10 | 30 | 26.6 | 80 |
| SR | SMX | 0 | 0 | 0 | 150 | 600 | 150 | 600 | 0 | 0 |
| | TMP | 0 | 0 | 0 | 30 | 90 | 30 | 90 | 0 | 0 |
| DR-3 | SMX | 0 | 0 | 0 | 0 | 0 | 0 | 0 | 133.3 | 533.3 |
| | TMP | 0 | 0 | 0 | 0 | 0 | 0 | 0 | 26.6 | 80 |

In general, the finished product can be manufactured in a variety of ways. The pulses can be combined to create from 1 up to as many as 5 individual units, dosed simultaneously. The individual pulses can be combined for simultaneous administration, in a package such as a sachet, capsule, etc., or compressed as one or more tablets to be administered simultaneously.

Numerous modification and variations of the present invention are possible in light of the above teachings and therefore within the scope of the appended claims the invention may be practiced otherwise than as particularly described. The present invention also extends to formulations which are bioequivalent to the pharmaceutical formulations of the present invention, in terms of both rate and extent of absorption, for instance as defined by the US Food and Drug Administration and discussed in the so-called “Orange Book” (Approved Drug Compositions with Therapeutic Equivalence Evaluations, US Dept of Health and Human Services, 19th edn, 1999).

We claim:

1. A once-a-day anti-MRSA antibiotic product comprising: a therapeutically-effective anti-MRSA daily dosage of a combination of at least three different antibiotics, wherein one of the at least three different antibiotics is selected from the group consisting of RNA-Polymerase Inhibiting antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; said daily dosage being the total dosage of said so selected antibiotic combination for a twenty-four hour period.

2. The product of claim 1, further comprising at least one component(s) selected from the group comprising immediate release components and modified release components; each component comprising a pharmaceutically acceptable carrier and at least one antibiotic.

3. The product of claim 2, wherein said modified release component(s) is/are selected from the group consisting of: delayed release component(s), sustained (or extended) release component(s), and combinations of the foregoing.

4. The product of claim 3, wherein said modified release component(s) is/are a delayed release component(s).

5. The product of claim 5, wherein said modified release component(s) is/are a sustained release component(s).

6. The product of claim 2, wherein said at least one antibiotic component(s) is/are an immediate release component(s).

7. The product of claim 1, wherein any of said RNA-Polymerase Inhibiting antibiotics, Dihydropteroate Synthase Inhibiting antibiotics, and Dihydrofolate Reductase Inhibiting antibiotics comprise analogues, derivatives, polymorphs, metabolites, pro-drugs, salts, and/or hydrates thereof.

8. The product of claim 7, wherein any of said RNA-Polymerase Inhibiting antibiotics, analogues, derivatives, polymorphs, metabolites, pro-drugs, salts, and/or hydrates thereof are selected from the group consisting of: rifamycin; rifampin; rifampin; rifabutin; rifapentin; rifapentine; rifaximin; rifalazil; rifaximin; the ansamycin antibiotics (drug class); rifamycin SV; rifamycin B diethylamide; rifamycin W; rifamycin S; rifamycin P; rifamycin O; rifamycin R; rifamycin U; rifamycin Y; rifamycin 3-iminomethylenyl (—CH═N—) derivatives; rifamycin-imino-derivatives; rifamycin-C11-oxime derivatives; rifamycin-C11-oxime cyclo derivatives; spiro-rifamycin; C-25 carbamate rifamycin derivatives; rifamexil; rifamdin; rifamide; rifaprim; rifamet(h)oprim; kanglemycin A; protorifamycin; rifamycin verde; ansamycin LM427; rifamazine; streptolygidin; sorangicin A; MDL473; GE23077; other (bacterial) RNA-Polymerase inhibitors such as the CBR703 series (Artsimovitch I, et al., Science. 2003 Oct. 24; 302(5645):650-4); Microcin J25 (Mukhopadhyay et al. 2004 Mol Cell 14:739; Adelman et al. 2004 Mol Cell 14:753); and any antibiotic which may act synergistically with any, or each, of a Dihydrofolate Reductase Inhibiting antibiotic, a Dihydropteroate Synthase Inhibiting antibiotic, or an any analogue (derivative etc.) of a Dihydrofolate Reductase Inhibiting antibiotic, or a Dihydropteroate Synthase Inhibiting antibiotic.

9. The product of claim 7, wherein any of said Dihydropteroate Synthase Inhibiting antibiotics, analogues, derivatives, polymorphs, metabolites, pro-drugs, salts, and/or hydrates thereof are selected from the group consisting of: sulfamethoxazole; para-aminobenzenesulfonamide derivatives (aka sulfanilamide-analogues); sulfanilamide; sulfadiazine; sulfamethoxazole; sulfadoxine; sulfisoxazole; sulfisoxazole acetyl; sulfacetmide; sulfapyridine; sulfasalazine; mafenide; sulfadimethoxine; sulfaquinoxaline; silver sulfadiazine; mafenide acetate; sulfisoxazole diolamine; phtalylsulfacetamide; phtalylsulfathiazole; sulfaguanidine; sulfamazole; sulfamazone; sulfametopirazine; sulfametoxypiridazine; sulfametrol; succinylsulfathiazole; 2,4-Diaminoquinazolines; Pyridopyrimidines; 2,3-a-pyrimidines; 5-deaza-pteridine; Epiroprim; Brodimoprim; K-130; SRI 8858; the class of the sulfones (aka diaminophenylsulfones, such as dapsone and sulfoxone); and any antibiotic which may act synergistically with any, or each, of a Dihydrofolate Reductase Inhibiting antibiotic, a RNA-Polymerase Inhibiting antibiotic, or an any analogue (derivative etc.) of a Dihydrofolate Reductase Inhibiting antibiotic, or a RNA-Polymerase Inhibiting antibiotic.

10. The product of claim 7, wherein any of said Dihydrofolate Reductase Inhibiting antibiotics, analogues, derivatives, polymorphs, metabolites, pro-drugs, salts, and/or hydrates thereof are selected from the group consisting of: trimethoprim, pyrimethamine; iclaprim; ormetoprim; 2,4-diaminopyrimidine; sulfanilic acid; nitrosoisocytosines; monocyclic pteridines; proguanil; chloroguanide; cycloguanil; and any antibiotic which may act synergistically with any, or each, of a Dihydropteroate Synthase Inhibiting antibiotic, a RNA-Polymerase Inhibiting antibiotic, or an any analogue (derivative etc.) of a Dihydropteroate Synthase Inhibiting antibiotic, or a RNA-Polymerase Inhibiting antibiotic.

11. The product of claim 1, wherein said combination of antibiotics are present in the product in the following amounts: about 20 mg. to about 2000 mg. of a RNA-Polymerase Inhibiting antibiotic, about 100 mg. to about 3000 mg. of a Dihydropteroate Synthase Inhibiting antibiotic, and about 20 mg. to about 2000 mg. of a Dihydrofolate Reductase Inhibiting antibiotic.

12. The product of claim 1, wherein said product is designed for oral administration.

13. The product of claim 1, further comprising at least one resistance inhibitor.

14. The product of claim 13, wherein said resistance inhibitor is selected from the group consisting of: any achaogen able to reduce the rate of induced mutagenesis, nucleic acids, peptide nucleic acids, phages, phagemids, polypeptides, peptidomimetics, antibodies, small or large organic or inorganic molecules, and combinations of the foregoing; whether of natural or non-natural origin; wherein they are able to bind to, or interact with, gene products that increase rate of mutations in a cell or organism; wherein such gene products are selected from the groups consisting of RecA, RecB, RecC, RecD, RecF, RecG, Rec N, LexA, UmuC, UmuD, PolB, PolIV, Poly, PriA, RuvA, RuvB, RuvC, UmuC, UmuD, UvrA, UvrB, UvrD, and homologs and analogs of the foregoing; and wherein said polypeptides are selected from the group consisting of dipeptide Ala-Ala, tripeptide Val-Ala-Ala, and SEQ ID NO: 1, 2, or 3.

15. The product of claim 13, wherein said at least one resistance inhibitor is a LexA protease cleavage inhibitor.

16. The product of claim 1, wherein said therapeutically-effective anti-MRSA daily dosage of said combination is an amount that is therapeutically-effective against infections caused by healthcare-acquired Methicillin-Resistant Staphylococcus aureus (HA-MRSA).

17. The product of claim 1, wherein said therapeutically-effective anti-MRSA daily dosage of said combination is an amount that is therapeutically-effective against infections caused by community-acquired Methicillin-Resistant Staphylococcus aureus (CA-MRSA).

18. The product of claim 1, wherein said therapeutically-effective anti-MRSA daily dosage of said combination is an amount that is therapeutically-effective for treating a patient suffering from a disease caused by a Methicillin-Resistant Staphylococcus aureus infection.

19. The product of claim 18, wherein said disease is selected from the group selected from: skin and soft tissue infections; boils; pimples; pneumonia, empyema, blood infections, bacteremia, sepsis, osteomyelitis, pyomytosis, necrotizing fascititis, purpura fulminans, infections of the bones and joints, urinary tract infections, toxic shock syndrome; and pluralities of the foregoing.

20. The product of claim 2, wherein said at least one component(s) comprises: an immediate release component containing rifampin in a range of about 150 mg. to about 600 mg.; an immediate release component containing a combination of sulfamethoxazole and trimethoprim (SMX-TMP), wherein the sulfamethoxazole is present in a range of about 200 mg. to about 800 mg. and the trimethoprim is present in a range of about 40 mg. to about 160 mg.; a first (in time to release) delayed release component containing a combination of sulfamethoxazole and trimethoprim (SMX-TMP), wherein the sulfamethoxazole is present in a range of about 100 mg. to about 400 mg. and the trimethoprim is present in a range of about 20 mg. to about 80 mg.; and a second (in time to release) delayed release component containing a combination of sulfamethoxazole and trimethoprim (SMX-TMP), wherein the sulfamethoxazole is present in a range of about 100 mg. to about 400 mg. and the trimethprim is present in a range of about 20 mg. to about 80 mg.

21. The product of claim 2, wherein said at least one component(s) comprises: an immediate release component containing rifampin in a range of about 150 mg. to about 600 mg.; an immediate release component containing a combination of sulfamethoxazole and trimethoprim (SMX-TMP), wherein the sulfamethoxazole is present in a range of about 200 mg. to about 800 mg. and the trimethoprim is present in a range of about 40 mg. to about 160 mg.; a delayed release component containing a combination of sulfamethoxazole and trimethoprim (SMX-TMP), wherein the sulfamethoxazole is present in a range of about 50 mg. to about 200 mg. and the trimethoprim is present in a range of about 10 mg. to about 30 mg.; and a sustained release component containing a combination of sulfamethoxazole and trimethoprim (SMX-TMP), wherein the sulfamethoxazole is present in a range of about 150 mg. to about 600 mg. and the trimethprim is present in a range of about 30 mg. to about 90 mg.

22. The product of claim 2, wherein said at least one component(s) comprises: an immediate release component containing rifampin in a range of about 150 mg. to about 600 mg.; a first (in time to release) delayed release component containing a combination of sulfamethoxazole and trimethoprim (SMX-TMP), wherein the sulfamethoxazole is present in a range of about 130 mg. to about 540 mg. and the trimethoprim is present in a range of about 20 mg. to about 80 mg.; a second (in time to release) delayed release component containing a combination of sulfamethoxazole and trimethoprim (SMX-TMP), wherein the sulfamethoxazole is present in a range of about 130 mg. to about 540 mg. and the trimethprim is present in a range of about 20 mg. to about 80 mg.; and a sustained release component containing a combination of sulfamethoxazole and trimethoprim (SMX-TMP), wherein the sulfamethoxazole is present in a range of about 130 mg. to about 540 mg. and the trimethprim is present in a range of about 20 mg. to about 80 mg.

23. A process for treating Methicillin-Resistant Staphylococcus Aureus infection in a host comprising: administering to a host the antibiotic product of claim 1, once-a-day.

24. A process for treating Methicillin-Resistant Staphylococcus Aureus infection in a host comprising: administering to a host the antibiotic product of claim 2, once-a-day.

25. A process for treating Methicillin-Resistant Staphylococcus Aureus infection in a host comprising: administering to a host the antibiotic product of claim 3, once-a-day.

26. A process for treating Methicillin-Resistant Staphylococcus Aureus infection in a host comprising: administering to a host the antibiotic product of claim 4, once-a-day.

27. A process for treating Methicillin-Resistant Staphylococcus Aureus infection in a host comprising: administering to a host the antibiotic product of claim 5, once-a-day.

28. A process for treating Methicillin-Resistant Staphylococcus Aureus infection in a host comprising: administering to a host the antibiotic product of claim 6, once-a-day.

29. A process for treating Methicillin-Resistant Staphylococcus Aureus infection in a host comprising: administering to a host the antibiotic product of claim 7, once-a-day.

30. A process for treating Methicillin-Resistant Staphylococcus Aureus infection in a host comprising: administering to a host the antibiotic product of claim 8, once-a-day.

31. A process for treating Methicillin-Resistant Staphylococcus Aureus infection in a host comprising: administering to a host the antibiotic product of claim 9, once-a-day.

32. A process for treating Methicillin-Resistant Staphylococcus Aureus infection in a host comprising: administering to a host the antibiotic product of claim 10, once-a-day.

33. A process for treating Methicillin-Resistant Staphylococcus Aureus infection in a host comprising: administering to a host the antibiotic product of claim 11, once-a-day.

34. A process for treating Methicillin-Resistant Staphylococcus Aureus infection in a host comprising: administering to a host the antibiotic product of claim 12, once-a-day.

35. A process for treating Methicillin-Resistant Staphylococcus Aureus infection in a host comprising: administering to a host the antibiotic product of claim 13, once-a-day.

36. A process for treating Methicillin-Resistant Staphylococcus Aureus infection in a host comprising: administering to a host the antibiotic product of claim 14, once-a-day.

37. A process for treating Methicillin-Resistant Staphylococcus Aureus infection in a host comprising: administering to a host the antibiotic product of claim 15, once-a-day.

38. A process for treating Methicillin-Resistant Staphylococcus Aureus infection in a host comprising: administering to a host the antibiotic product of claim 16, once-a-day.

39. A process for treating Methicillin-Resistant Staphylococcus Aureus infection in a host comprising: administering to a host the antibiotic product of claim 17, once-a-day.

40. A process for treating Methicillin-Resistant Staphylococcus Aureus infection in a host comprising: administering to a host the antibiotic product of claim 18, once-a-day.

41. A process for treating Methicillin-Resistant Staphylococcus Aureus infection in a host comprising: administering to a host the antibiotic product of claim 19, once-a-day.

42. A process for treating Methicillin-Resistant Staphylococcus Aureus infection in a host comprising: administering to a host the antibiotic product of claim 20, once-a-day.

43. A process for treating Methicillin-Resistant Staphylococcus Aureus infection in a host comprising: administering to a host the antibiotic product of claim 21, once-a-day.

44. A process for treating Methicillin-Resistant Staphylococcus Aureus infection in a host comprising: administering to a host the antibiotic product of claim 22, once-a-day.

45. The product of claim 1, wherein said RNA-Polymerase Inhibiting antibiotic is saphenamycin.

46. A process for treating Methicillin-Resistant Staphylococcus Aureus infection in a host comprising: administering to a host the antibiotic product of claim 45, once-a-day.

47. A once-a-day anti-MRSA antibiotic product comprising: a therapeutically-effective anti-MRSA daily dosage of a combination of at least three different antibiotics, wherein one of the at least three different antibiotics is selected from the group consisting of Phenazine antibiotics, wherein one of the at least three different antibiotics is selected from the group consisting of Dihydropteroate Synthase Inhibiting antibiotics, and wherein one of the at least three different antibiotics is selected from the group consisting of Dihydrofolate Reductase Inhibiting antibiotics; said daily dosage being the total dosage of said so selected antibiotic combination for a twenty-four hour period.

48. A process for treating Methicillin-Resistant Staphylococcus Aureus infection in a host comprising: administering to a host the antibiotic product of claim 47, once-a-day.

49. The product of claim 47, wherein said Phenazine antibiotic is saphenamycin.

50. A process for treating Methicillin-Resistant Staphylococcus Aureus infection in a host comprising: administering to a host the antibiotic product of claim 49, once-a-day.


SEQUENCE LISTING (3 sequences)

SEQ ID NO: 1 - protein, 4 aa, Artificial
  Synthetic peptide corresponding to LexA cleavage site
VAAG

SEQ ID NO: 2 - protein, 7 aa, Artificial
  Synthetic peptide corresponding to LexA cleavage site
VAAGEPL

SEQ ID NO: 3 - protein, 9 aa, Artificial
  Synthetic peptide corresponding to LexA cleavage site
VAGEPLLAW